(19) United States
(12) Patent Application Publication
(10) Pub. No.: US 2016/0007606 A1
(43) Pub. Date: Jan. 14, 2016

(54) MICROBIOCIDAL PYRAZOLE DERIVATIVES

(71) Applicant: SYNGENTA PARTICIPATIONS AG, Basel (CH)

(72) Inventors: Clemens LAMBERTH, Stein (CH); Sarah SULZER-MOSSE, Stein (CH); Fredrik CEDERBAUM, Stein (CH); Jayant UMARYE, Goa (IN); Ravindra SONAWANE, Goa (IN)

(73) Assignee: SYNGENTA PARTICIPATIONS AG, Basel (CH)

(21) Appl. No.: 14/379,003

(22) PCT Filed: Feb. 26, 2013
(86) PCT No.: PCT/EP13/53803

§ 371 Date: Aug. 15, 2014

(30) Foreign Application Priority Data
Mar. 2, 2012 (IN) 620/DELNP/2012

(51) Int. Cl. A01N 43/82; A01N 43/76; C07D 417/14; A01N 43/80; A01N 43/78; C07D 413/14

(52) U.S. Cl. CPC A01N 43/82; A01N 43/78; C07D 413/14; C07D 417/14; A01N 43/80; A01N 43/76

(57) ABSTRACT

Compounds of the formula (I) wherein the substituents are as defined in claim 1, are useful as a pesticides.

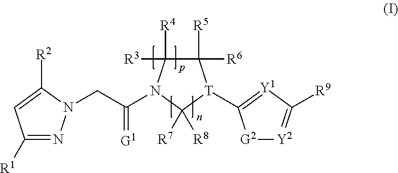

The present invention relates to microbiocidal pyrazole derivatives, e.g. as active ingredients, which have microbiocidal activity, in particular fungicidal activity. The invention also relates to preparation of these pyrazole derivatives, to pyrazole derivatives used as intermediates in the preparation of these pyrazole derivatives, to preparation of these intermediates, to agrochemical compositions which comprise at least one of the pyrazole derivatives, to preparation of these compositions and to use of the pyrazole derivatives or compositions in agriculture or horticulture for controlling or preventing infestation of plants, harvested food crops, seeds or non-living materials by phytopathogenic microorganisms, preferably fungi.

Certain compounds for use as fungicides are described in WO 2007/014290, WO 2008/013622, WO 2008/013925, WO 2008/091580, WO 2008/091594 and WO 2009/055514.

The present invention provides compounds of formula I:

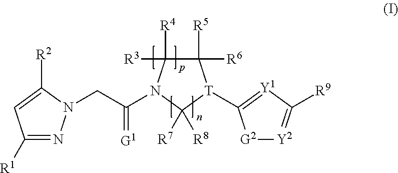

wherein,
G¹ and G² are independently O or S;

### T is CR¹⁰ or N;

Y¹ and Y² are independently CR¹¹ or N;
n is 1 or 2;
p is 1 or 2, providing that when n is 2, p is 1;
R¹ and R² each independently are C₁-C₄alkyl, C₃-C₅cycloalkyl or C₁-C₄haloalkyl;
R³, R⁴, R⁵, R⁶, R⁷ and R⁸ each independently are hydrogen, C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl, providing that at least one of R³, R⁴, R⁵, R⁶, R⁷ or R⁸ is C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl;
R⁹ is C(═O)—OR¹², C(═O)—NR¹²R¹³ or a 4- to 15-membered mono-, bi- or tricyclic, saturated, partially unsaturated or aromatic heterocyclic ringsystem optionally substituted by one or more R¹⁴;
R¹⁰ is hydrogen, halogen or hydroxyl;
R¹¹ is hydrogen, halogen or cyano;
R¹² is arylalkyl, heteroarylalkyl or a 4- to 11-membered mono- or bicyclic, saturated, partially unsaturated or aromatic carbocyclic ringsystem, wherein the arylalkyl, heteroarylalkyl and 4- to 11-membered carbocyclic ringsystem are optionally substituted by one or more R¹⁴;
R¹³ is hydrogen, C₁-C₄alkyl, C₃-C₅cycloalkyl or C₁-C₄alkoxy;
each R¹⁴ independently is halogen, cyano, amino, nitro, hydroxyl, mercapto, C₁-C₈alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkoxy, C₃-C₈cycloalkyl-C₁-C₄alkylthio, C₁-C₈alkoxy, C₃-C₈cycloalkoxy, C₁-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkoxy, aryl-C₁-C₄alkylthio, heterocyclyl, heterocycyl-C₁-C₄alkyl, heterocycyl-C₁-C₄alkoxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R¹⁵; and
each R¹⁵ independently is halogen, cyano, C₁-C₄alkyl, C₁-C₄haloalkyl, C₁-C₄alkoxy or C₁-C₄haloalkoxy; or a salt or a N-oxide thereof.

For the avoidance of doubt, by the expression “providing that at least one of R³, R⁴, R⁵, R⁶, R⁷ or R⁸ is C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl”, it is intended herein that at least one of R³, R⁴, R⁵, R⁶, R⁷ or R⁸ is not hydrogen.

Where substituents are indicated as being optionally substituted, this means that they may or may not carry one or more identical or different substituents, e.g. one to five substituents, e.g. one to three substituents. Normally not more than three such optional substituents are present at the same time. Where a group is indicated as being substituted, e.g. alkyl, unless stated otherwise this includes those groups that are part of other groups, e.g. the alkyl in alkylthio.

The term “halogen” refers to fluorine, chlorine, bromine or iodine, preferably fluorine, chlorine or bromine.

Alkyl substituents may be straight-chained or branched. Alkyl on its own or as part of another substituent is, depending upon the number of carbon atoms mentioned, for example, methyl, ethyl, n-propyl, n-butyl, n-pentyl, n-hexyl and the isomers thereof, for example, iso-propyl, iso-butyl, sec-butyl, tert-butyl, iso-amyl or pivaloyl.

Alkenyl substituents can be in the form of straight or branched chains, and the alkenyl moieties, where appropriate, can be of either the (E)- or (Z)-configuration. Examples are vinyl and allyl. The alkenyl groups are preferably C₂-C₆, more preferably C₂-C₄ and most preferably C₂-C₃ alkenyl groups.

Alkynyl substituents can be in the form of straight or branched chains. Examples are ethynyl and propargyl. The alkynyl groups are preferably C₂-C₆, more preferably C₂-C₄ and most preferably C₂-C₃ alkynyl groups.

Haloalkyl groups may contain one or more identical or different halogen atoms and, for example, may stand for CH₂Cl, CHCl₂, CCI₃, CH₂F, CHF₂, CF₃, CF₃CH₂, CH₃CF₂, CF₃CF₂ or CCl₃CCl₂.

Haloalkenyl groups are alkenyl groups, respectively, which are substituted with one or more of the same or different halogen atoms and are, for example, 2,2-difluorovinyl or 1,2-dichloro-2-fluoro-vinyl.

Haloalkynyl groups are alkynyl groups, respectively, which are substituted with one or more of the same or different halogen atoms and are, for example, 1-chloro-prop-2-ynyl.

Alkoxy means a radical —OR, where R is alkyl, e.g. as defined above. Alkoxy groups include, but are not limited to, methoxy, ethoxy, 1-methylethoxy, propoxy, butoxy, 1-methylpropoxy and 2-methylpropoxy.

Cyano means a —CN group.

Amino means an NH₂ group.

Hydroxyl or hydroxy stands for a —OH group.

Aryl means a ring system which may be mono-, bi- or tricyclic. Examples of such rings include phenyl, naphthalenyl, anthracenyl, indenyl or phenanthrenyl. A preferred aryl group is phenyl.

Heteroaryl stands for aromatic ring systems comprising mono-, bi- or tricyclic systems wherein at least one oxygen, nitrogen or sulfur atom is present as a ring member. Monocyclic and bicyclic aromatic ring systems are preferred, monocyclic ring systems are more preferred. For example, monocyclic heteoraryl may be a 5- to 7-membered aromatic ring containing one to three heteroatoms selected from oxygen, nitrogen and sulfur, more preferably selected from nitrogen and sulfur. Bicyclic heteroaryl may be a 9- to 11-membered bicyclic ring containing one to five heteroatoms, preferably one to three heteroatoms, selected from oxygen, nitrogen and sulfur. Examples are furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyridazinyl, pyrimidinyl, pyrazinyl, triazinyl, tetrazinyl, indolyl, benzothiophenyl, benzofuranyl, benzimidazolyl, indazolyl, benzotriazolyl, benzothiazolyl, benzoxazolyl, imiazothiazoyl, quinolinyl, quinoxalinyl, isoquinolinyl, phthalazinyl, quinoxalinyl, quinazolinyl, cinnolinyl and naphthyridinyl, preferably pyridyl, pyrazinyl, pyridazinyl, pyrimidinyl, pyrrolyl, pyrazolyl, imidazolyl, triazolyl, furanyl, thienyl thiazolyl or thiadiazolyl. Heteroaryl rings do not contain adjacent oxygen ring atoms, adjacent sulfur ring atoms or adjacent oxygen and sulfur ring atoms. A link to a heteroaryl group can be via a carbon atom or via a nitrogen atom.

Heterocyclyl is defined to include heteroaryl and in addition their saturated or partially unsaturated analogues. The different rings of bi- or tricyclic heterocyclic ring systems may be linked via one atom belonging to two different rings (spiro), via two adjacent ring atoms belonging to two different rings (annelated) or via two different, not adjacent ring atoms belonging to two different rings (bridged).

The presence of one or more possible asymmetric carbon atoms in a compound of formula I means that the compounds may occur in optically isomeric forms, i.e. enantiomeric or diastereomeric forms. Also atropisomers may occur as a result of restricted rotation about a single bond. Formula I is intended to include all those possible isomeric forms and mixtures thereof. The present invention includes all those possible isomeric forms and mixtures thereof for a compound of formula I. Likewise, formula I is intended to include all possible tautomers. The present invention includes all possible tautomeric forms for a compound of formula I.

In each case, the compounds of formula I according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The following list provides definitions, including preferred definitions, for substituents G¹, G², T, Y¹, Y², n, p, R¹, R², R³, R⁴, R⁵, R⁶, R⁷, R⁸, R⁹, R¹⁰, R¹¹, R¹², R¹³, R¹⁴ and R¹⁵ with reference to compounds of formula I and other compounds of the invention carrying the same substituents. For any one of these substituents, any of the definitions given below may be combined with any definition of any other substituent given below or elsewhere in this document.

G¹ and G² are independently 0 or S.

G¹ is preferably O.

G² is preferably S.

T is CR¹⁰ or N, preferably CH or N, more preferably CH.

Y¹ and Y² are independently CR¹¹ or N.

Y¹ is preferably CH or N, more preferably N.

Y² is preferably CH or N; more preferably CH.

n is 1 or 2, preferably 2.

p is 1 or 2, providing that when n is 2, p is 1, preferably p is 1.

R¹ and R² each independently are C₁-C₄alkyl, C₃-C₅cycloalkyl or C₁-C₄haloalkyl. Preferably R¹ and R² are each independently methyl or halomethyl, more preferably methyl, difluoromethyl or trifluoromethyl.

Preferably R¹ is difluoromethyl or trifluoromethyl. Preferably R² is methyl or difluoromethyl. In one group of compounds R¹ is trifluoromethyl and R² is methyl. In another group of compounds R¹ and R² are both difluoromethyl.

R³, R⁴, R⁵, R⁶, R⁷ and R⁸ each independently are hydrogen, C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl, providing that at least one of R³, R⁴, R⁵, R⁶, R⁷ or R⁸ is C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl.

Preferably one of R³ and R⁵ is carboxyl, C₁-C₄alkoxycarbonyl, C₁-C₄haloalkoxycarbonyl, aminocarbonyl, (C₁-C₄alkyl)aminocarbonyl, (C₁-C₄haloalkyl)aminocarbonyl, (C₁-C₄alkyl)₂aminocarbonyl or (C₁-C₄haloalkyl)₂aminocarbonyl and R⁴, R⁶, R⁷ and R⁸ are hydrogen;

More preferably one of R³ and R⁵ is carboxyl, C₁-C₄alkoxycarbonyl, aminocarbonyl, (C₁-C₄alkyl)aminocarbonyl, (C₁-C₄alkyl)₂aminocarbonyl and R⁴, R⁶, R⁷ and R⁸ are hydrogen.

Even more preferably, one of R³ and R⁵ is carboxyl or C₁-C₄alkoxycarbonyl and R⁴, R⁶, R⁷ and R⁸ are hydrogen.

R⁹ is C(═O)—OR¹², C(═O)—NR¹²R¹³ or a 4- to 15-membered mono-, bi- or tricyclic, saturated, partially unsaturated or aromatic heterocyclic ringsystem optionally substituted by one or more R¹⁴. Preferably R⁹ is C(═O)—NR¹²R¹³ or a 5-membered heterocyclic ringsystem optionally substituted by one or more R¹⁴. More preferably R⁹ is C(═O)—NR¹²R¹³ or a isoxazoline optionally substituted by one or more R¹⁴.

R¹⁰ is hydrogen, halogen or hydroxyl; preferably hydrogen, fluoro or hydroxyl, more preferably hydrogen.

R¹¹ is hydrogen, halogen or cyano; preferably hydrogen or cyano, more preferably hydrogen.

R¹² is arylalkyl, heteroarylalkyl or a 4- to 11-membered mono- or bicyclic, saturated, partially unsaturated or aromatic carbocyclic ringsystem, wherein the arylalkyl, heteroarylalkyl and 4- to 11-membered carbocyclic ringsystem are optionally substituted by one or more R¹⁴. Preferably R¹² is arylalkyl or a 9- to 10-membered bicyclic carbocyclic ringsystem, wherein the arylalkyl and 9- to 10-membered bicyclic carbocyclic ringsystem are optionally substituted by one or more R¹⁴. More preferably R¹² is benzyl or a 10-membered bicyclic carbocyclic ringsystem, wherein the benzyl and 10-membered bicyclic carbocyclic ringsystem are optionally substituted by one or more R¹⁴.

R¹³ is hydrogen, C₁-C₄alkyl, C₃-C₅cycloalkyl or C₁-C₄alkoxy. Preferably R¹³ is hydrogen or C₁-C₄alkyl. More preferably R¹³ is hydrogen or methyl.

Each R¹⁴ is independently, halogen, cyano, amino, nitro, hydroxyl, mercapto, C₁-C₈ alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkyloxy, C₃-C₈cycloalkyl-C₁-C₄alkylthio, C₁-C₈alkoxy, C₃-C₈cycloalkyloxy, C₂-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkoxy, aryl-C₁-C₈alkylthio, heterocyclyl, heterocycyl-C₁-C₄alkyl, heterocycyl-C₁-C₄alkoxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein the aryl and heterocyclyl are optionally substituted by one or more R¹⁵.

Preferably each R¹⁴ independently is halogen, cyano, amino, nitro, hydroxyl, mercapto, C₁-C₈ alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkylthio, C₁-C₈alkoxy, C₃-C₈cycloalkoxy, C₂-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, phenyl, phenyloxy, phenylthio, phenylsulfonyl, phenylsulfinyl, phenyl-C₁-C₄alkyl, phenyl-C₁-C₄alkyloxy, phenyl-C₁-C₄alkylthio, heterocyclyl, heterocycyl-C₁-C₄alkyl, heterocycyl-C₁-C₄alkyloxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R¹⁵; and wherein heterocyclyl is independently selected from pyridyl, pyrazinyl, pyridazinyl, pyrimidinyl, pyrrolyl, pyrazolyl, imidazolyl, triazolyl, furanyl, thienyl, thiazolyl, thiadiazolyl, pyrrolidinyl, piperazinyl, piperidinyl, morpholinyl, and tetrahydropyranyl.

More preferably each R¹⁴ independently is halogen, cyano, amino, mercapto, C₁-C₈alkyl, C₃-C₈cycloalkyl, C₃-C₈cycloalkyl-C₁-C₄alkyloxy, C₃-C₈cycloalkyl-C₁-C₄alkylthio, C₁-C₈alkoxy, C₁-C₈alkylthio, phenyl, phenyloxy, phenylthio, phenyl-C₁-C₄alkoxy, phenyl-C₁-C₄alkylthio, heterocyclyl, heterocyclyl-C₁-C₄alkoxy, heterocyclyl-C₁-C₄alkylthio, NH(C₁-C₈alkyl), N(C₁-C₈alkyl)₂, and wherein heterocyclyl is indepedendently selected from pyridyl, pyrazinyl, pyridazinyl, pyrimidinyl, pyrrolyl, pyrazolyl, imidazolyl, triazolyl, furanyl, thienyl, thiazolyl, thiadiazolyl, pyrrolidinyl, piperazinyl, piperidinyl, morpholinyl, and tetrahydroyranyl, and wherein alkyl, cycloalkyl and alkoxy are optionally substituted by halogen, and wherein aryl and heterocyclyl moieties are optionally substituted by one or more R¹⁵.

Even more preferably each R¹⁴ independently is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁵.

Each R¹⁵ is independently halogen, cyano, C₁-C₄alkyl, C₁-C₄haloalkyl, C₁-C₄alkoxy or C₁-C₄haloalkoxy, preferably each R¹⁵ independently is halogen, cyano, methyl, halomethyl, methoxy or halomethoxy, more preferably each R¹⁵ independently is halogen, methyl or halomethyl.

The compound of the invention may be a compound of formula I-a

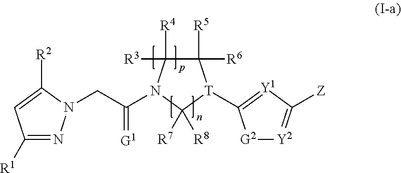

wherein Z is selected from Z-1 to Z-12

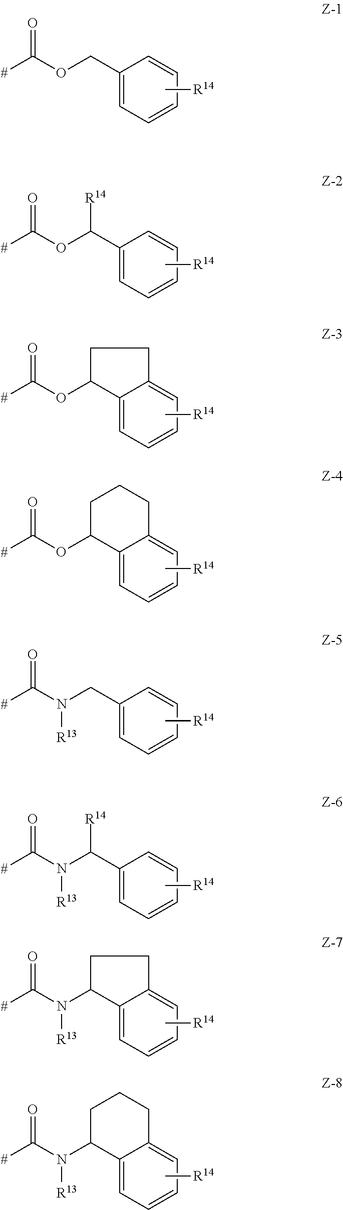

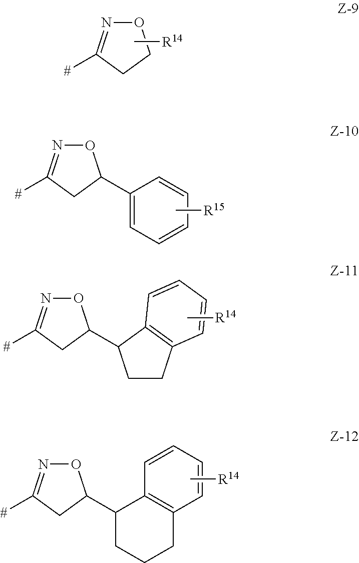

and G¹, G², T, Y¹, Y², n, p, R¹, R², R³, R⁴, R⁵, R⁶, R⁷, R⁸, R⁹, R¹⁰, R¹¹, R¹², R¹³, R¹⁴ and R¹⁵ are as defined for formula I. The preferred substituent definitions are the same as for compounds of formula I. Preferably Z is selected from Z-1, Z-4, Z-8, Z-10 and Z-12.

Preferably, in compounds according to formula I-a, R¹⁴ and R¹⁵ are independently C₁-C₄alkyl, C₁-C₄haloalkyl or halogen.

In one group of compounds of the invention G¹ and G² are independently O or S; T is CR¹⁰ or N; Y¹ is N; Y² is CR¹¹ or N; n is 1 or 2; p is 1; R¹ and R² each independently are C₁-C₄alkyl, C₃-C₅cycloalkyl or C₁-C₄haloalkyl; one of R³ and R⁵ is carboxyl, C₁-C₄alkoxycarbonyl, C₁-C₄haloalkoxycarbonyl, aminocarbonyl, (C₁-C₄alkyl)aminocarbonyl, (C₁-C₄haloalkyl)aminocarbonyl, (C₁-C₄alkyl)₂aminocarbonyl or (C₁-C₄haloalkyl)₂aminocarbonyl; R⁴, R⁶, R⁷ and R⁸ are hydrogen; R⁹ is C(═O)—OR¹², C(═O)—NR¹²R¹³ or a 4- to 15-membered mono-, bi- or tricyclic, saturated, partially unsaturated or aromatic heterocyclic ringsystem optionally substituted by one or more R¹⁴; R¹⁰ is hydrogen, halogen or hydroxyl; R¹¹ is hydrogen, halogen or cyano; R¹² is arylalkyl, heteroarylalkyl or a 4- to 11-membered mono- or bicyclic, saturated, partially unsaturated or aromatic carbocyclic ringsystem, wherein the arylalkyl, heteroarylalkyl and 4- to 11-membered carbocyclic ringsystem are optionally substituted by one or more R¹⁴; R¹³ is hydrogen, C₁-C₄alkyl, C₃-C₅cycloalkyl or C₁-C₄alkoxy; each R¹⁴ independently is halogen, cyano, amino, nitro, hydroxyl, mercapto, C₁-C₈alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkoxy, C₃-C₈cycloalkyl-C₁-C₄alkylthio, C₁-C₈alkoxy, C₃-C₈cycloalkoxy, C₁-C₈alkenyloxy, C₂-C₈alkynyloxy, C₁-C₈alkylthio, C₁-C₈alkylsulfonyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkoxy, aryl-C₁-C₄alkylthio, heterocyclyl, heterocycyl-C₁-C₄alkyl, heterocycyl-C₁-C₄alkoxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R¹⁵; and

each R¹⁵ independently is halogen, cyano, C₁-C₄alkyl, C₁-C₄haloalkyl, C₁-C₄alkoxy or C₁-C₄haloalkoxy.

In another group of compounds of the invention G¹ is O; G² is S; T is CH or N; Y′ is N; Y² is CH; n is 2; p is 1; R¹ is difluoromethyl or trifluoromethyl; R² is methyl or difluoromethyl; R³ is C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl; R⁴, R⁵, R⁶, R⁷ and R⁸ are hydrogen; R⁹ is C(═O)—NR¹²R¹³ or a 5-membered heterocyclic ringsystem optionally substituted by one or more R¹⁴; R¹² is arylalkyl or a 9- to 10-membered bicyclic carbocyclic ringsystem, wherein the arylalkyl and 9- to 10-membered bicyclic carbocyclic ringsystem are optionally substituted by one or more R¹⁴; R¹³ is hydrogen or methyl; each R¹⁴ is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁵ and R¹⁵ is halogen, methyl or halomethyl.

In another group of compounds of the invention G¹ is O; G² is S; T is CH or N; Y¹ is N; Y² is CH; n is 2; p is 1; R¹ is difluoromethyl or trifluoromethyl; R² is methyl or difluoromethyl; R³ is carboxyl, C₁-C₄alkoxycarbonyl, C₁-C₄haloalkoxycarbonyl, aminocarbonyl, (C₁-C₄alkyl)aminocarbonyl, (C₁-C₄haloalkyl)aminocarbonyl, (C₁-C₄alkyl)₂aminocarbonyl or (C₁-C₄haloalkyl)₂aminocarbonyl; R⁴, R⁵, R⁶, R⁷ and R⁸ are hydrogen; R⁹ is C(═O)—NR¹²R¹³ or a 5-membered heterocyclic ringsystem optionally substituted by one or more R¹⁴; R¹² is arylalkyl or a 9- to 10-membered bicyclic carbocyclic ringsystem, wherein the arylalkyl and 9- to 10-membered bicyclic carbocyclic ringsystem are optionally substituted by one or more R¹⁴; R¹³ is hydrogen or methyl; each R¹⁴ is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁵ and R¹⁵ is halogen, methyl or halomethyl.

In another group of compounds of the invention G¹ is O; G² is S; T is CH or N; Y¹ is N; Y² is CH; n is 2; p is 1; R¹ is difluoromethyl or trifluoromethyl; R² is methyl or difluoromethyl; R³ is carboxyl, C₁-C₄alkoxycarbonyl, aminocarbonyl, (C₁-C₄alkyl)aminocarbonyl, or (C₁-C₄alkyl)₂aminocarbonyl; R⁴, R⁵, R⁶, R⁷ and R⁸ are hydrogen; R⁹ is C(═O)—NR¹²R¹³ or a 5-membered heterocyclic ringsystem optionally substituted by one or more R¹⁴; R¹² is arylalkyl or a 9- to 10-membered bicyclic carbocyclic ringsystem, wherein the arylalkyl and 9- to 10-membered bicyclic carbocyclic ringsystem are optionally substituted by one or more R¹⁴; R¹³ is hydrogen or methyl; each R¹⁴ is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁵ and R¹⁵ is halogen, methyl or halomethyl.

In another group of compounds of the invention G¹ is O; G² is S; T is CH or N; Y¹ is N; Y₂ is CH; n is 2; p is 1; R¹ is difluoromethyl or trifluoromethyl; R² is methyl or difluoromethyl; R⁵ is C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₆cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl; R³, R⁴, R⁶, R⁷ and R⁸ are hydrogen; R⁹ is C(═O)—NR¹²R¹³ or a 5-membered heterocyclic ringsystem optionally substituted by one or more R¹⁴; R¹² is arylalkyl or a 9- to 10-membered bicyclic carbocyclic ringsystem, wherein the arylalkyl and 9- to 10-membered bicyclic carbocyclic ringsystem are optionally substituted by one or more R¹⁴; R¹³ is hydrogen or methyl; each R¹⁴ is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁵ and R¹⁵ is halogen, methyl or halomethyl.

In another group of compounds of the invention G¹ is O; G² is S; T is CH or N; Y¹ is N; Y² is CH; n is 2; p is 1; R¹ is difluoromethyl or trifluoromethyl; R² is methyl or difluoromethyl; R⁵ is carboxyl, C₁-C₄alkoxycarbonyl, C₁-C₄haloalkoxycarbonyl, aminocarbonyl, (C₁-C₄alkyl)aminocarbonyl, (C₁-C₄haloalkyl)aminocarbonyl, (C₁-C₄alkyl)₂aminocarbonyl or (C₁-C₄haloalkyl)₂aminocarbonyl; R³, R⁴, R⁶, R⁷ and R⁸ are hydrogen; R⁹ is C(═O)—NR¹²R¹³ or a 5-membered heterocyclic ringsystem optionally substituted by one or more R¹⁴; R¹² is arylalkyl or a 9- to 10-membered bicyclic carbocyclic ringsystem, wherein the arylalkyl and 9- to 10-membered bicyclic carbocyclic ringsystem are optionally substituted by one or more R¹⁴; R¹³ is hydrogen or methyl; each R¹⁴ is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁵ and R¹⁵ is halogen, methyl or halomethyl.

In another group of compounds of the invention G¹ is O; G² is S; T is CH or N; Y¹ is N; Y² is CH; n is 2; p is 1; R¹ is difluoromethyl or trifluoromethyl; R² is methyl or difluoromethyl; R⁵ is carboxyl, C₁-C₄alkoxycarbonyl, aminocarbonyl, (C₁-C₄alkyl)aminocarbonyl, or (C₁-C₄alkyl)₂aminocarbonyl; R³, R⁴, R⁶, R⁷ and R⁸ are hydrogen; R⁹ is C(═O)—NR¹²R¹³ or a 5-membered heterocyclic ringsystem optionally substituted by one or more R¹⁴; R¹² is arylalkyl or a 9- to 10-membered bicyclic carbocyclic ringsystem, wherein the arylalkyl and 9- to 10-membered bicyclic carbocyclic ringsystem are optionally substituted by one or more R¹⁴; R¹³ is hydrogen or methyl; each R¹⁴ is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R¹⁵ and R¹⁵ is halogen, methyl or halomethyl.

In another group of compounds the compound of the invention is a compound of formula I.a, wherein G¹ is O; G² is S; T is CH or N; Y¹ is N; Y² is CH; Z is selected from Z-1 to Z-12 (above); preferably Z-1, Z-4, Z-8, Z-10 and Z-12; n is 2; p is 1; R¹ is difluoromethyl or trifluoromethyl; R² is methyl or difluoromethyl; R³ is C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl and R⁴, R⁵, R⁶, R⁷ and R⁸ are hydrogen.

In another group of compounds the compound of the invention is a compound of formula I.a, wherein G¹ is O; G² is S; T is CH or N; Y¹ is N; Y² is CH; Z is selected from Z-1 to Z-12 (above); preferably Z-1, Z-4, Z-8, Z-10 and Z-12; n is 2; p is 1; R¹ is difluoromethyl or trifluoromethyl; R² is methyl or difluoromethyl; R³ is carboxyl, C₁-C₄alkoxycarbonyl, C₁-C₄haloalkoxycarbonyl, aminocarbonyl, (C₁-C₄alkyl)aminocarbonyl, (C₁-C₄haloalkyl)aminocarbonyl, (C₁-C₄alkyl)₂aminocarbonyl or (C₁-C₄haloalkyl)₂aminocarbonyl; and R⁴, R⁵, R⁶, R⁷ and R⁸ are hydrogen.

In another group of compounds the compound of the invention is a compound of formula I.a, wherein G¹ is O; G² is S; T is CH or N; Y¹ is N; Y² is CH; Z is selected from Z-1 to Z-12 (above); preferably Z-1, Z-4, Z-8, Z-10 and Z-12; n is 2; p is 1; R¹ is difluoromethyl or trifluoromethyl; R² is methyl or difluoromethyl; R³ is carboxyl, C₁-C₄alkoxycarbonyl, aminocarbonyl, (C₁-C₄alkyl)aminocarbonyl, or (C₁-C₄alkyl)₂aminocarbonyl; and R⁴, R⁵, R⁶, R⁷ and R⁸ are hydrogen.

In another group of compounds the compound of the invention is a compound of formula I.a, wherein G¹ is O; G² is S; T is CH or N; Y¹ is N; Y² is CH; Z is selected from Z-1 to Z12 (above); preferably Z-1, Z-4, Z-8, Z-10 and Z-12; n is 2; p is 1; R¹ is difluoromethyl or trifluoromethyl; R² is methyl or difluoromethyl; R⁵ is C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl and R³, R⁴, R⁶, R⁷ and R⁸ are hydrogen.

In another group of compounds the compound of the invention is a compound of formula I.a, wherein G¹ is O; G² is S; T is CH or N; Y¹ is N; Y² is CH; Z is selected from Z-1 to Z-12 (above); preferably Z-1, Z-4, Z-8, Z-10 and Z-12; n is 2; p is 1; R¹ is difluoromethyl or trifluoromethyl; R² is methyl or difluoromethyl; R⁵ is carboxyl, C₁-C₄alkoxycarbonyl, C₁-C₄haloalkoxycarbonyl, aminocarbonyl, (C₁-C₄alkyl)aminocarbonyl, (C₁-C₄haloalkyl)aminocarbonyl, (C₁-C₄alkyl)₂aminocarbonyl or (C₁-C₄haloalkyl)₂aminocarbonyl; and R³, R⁴, R⁶, R⁷ and R⁸ are hydrogen.

In another group of compounds the compound of the invention is a compound of formula I.a, wherein G¹ is O; G² is S; T is CH or N; Y¹ is N; Y² is CH; Z is selected from Z-1 to Z-12 (above); preferably Z-1, Z-4, Z-8, Z-10 and Z-12; n is 2; p is 1; R¹ is difluoromethyl or trifluoromethyl; R² is methyl or difluoromethyl; R⁵ is carboxyl, C₁-C₄alkoxycarbonyl, aminocarbonyl, (C₁-C₄alkyl)aminocarbonyl, or (C₁-C₄alkyl)₂aminocarbonyl; and R³, R⁴, R⁶, R⁷ and R⁸ are hydrogen.

For the avoidance of doubt, when n is 1 and p is 1 compounds of formula I have the formula according to formula I-b:

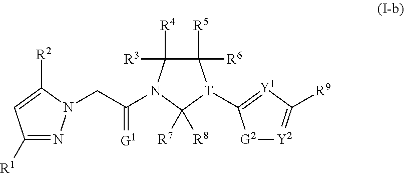

in which R¹, R², R³, R⁴, R⁵, R⁶, R⁷, R⁸, R⁹, G¹, G², T, Y¹ and Y² have the definitions as described for formula I.

When n is 2 and p is 1, compounds of formula I have the formula according to formula I-c:

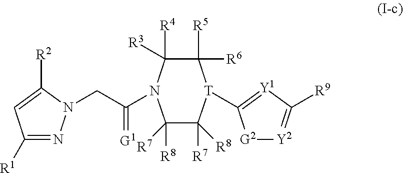

in which R¹, R², R³, R⁴, R⁵, R⁶, R⁷, R⁸, R⁹, G¹, G², T, Y¹ and Y² have the definitions as described for formula I.

When n is 1 and p is 2, compounds of formula I have the formula according to formula I-d:

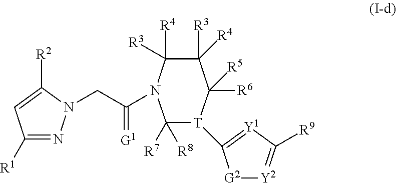

in which R¹, R², R³, R⁴, R⁵, R⁶, R⁷, R⁸, R⁹, G¹, G², T, Y¹ and Y² have the definitions as described for formula I.

The invention also relates to compounds of formula I-b, formula I-c and formula l-d as shown above.

The invention also relates to compounds of formula I-e:

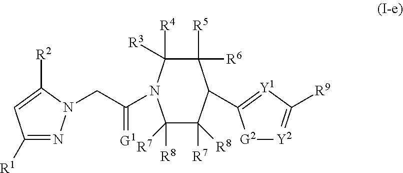

in which R¹, R², R³, R⁴, R⁵, R⁶, R⁷, R⁸, R⁹, G², Y¹ and Y² have the definitions as described for formula I. Preferred definitions of R¹, R², R³, R⁴, R⁵, R⁶, R⁷, R⁸, R⁹, G², Y¹ and Y² are as defined for formula I.

The invention also relates to compounds of formula I-f:

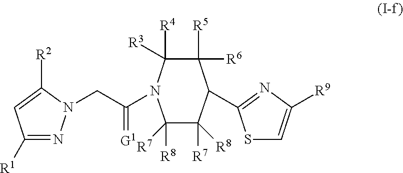

in which R¹, R², R³, R⁴, R⁵, R⁶, R⁷, R⁸ and R⁹ have the definitions as described for formula I. Preferred definitions of R¹, R², R³, R⁴, R⁵, R⁶, R⁷, R⁸ and R⁹ are as defined for formula I.

The invention also relates to compounds of formula I-g:

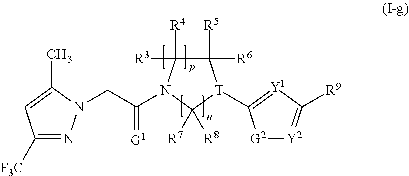

wherein R³, R⁴, R⁵, R⁶, R⁷, R⁸, R⁹, G¹, G², T, Y¹, Y², n and p have the definition as described for formula I. Preferred definitions of R³, R⁴, R⁵, R⁶, R⁷, R⁸, R⁹, G¹, G², T, Y¹, Y², n and p are as defined for formula I.

The invention also relates to compounds of formula I-h:

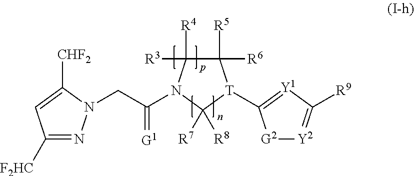

in which R³, R⁴, R⁵, R⁶, R⁷, R⁸, R⁹, G¹, G², T, Y¹, Y², n and p have the definitions as described for formula I. Preferred definitions of R³, R⁴, R⁵, R⁶, R⁷, R⁸, R⁹, G¹, G², T, Y¹, Y², n and p are as defined for formula I.

The invention includes compounds of formula II:

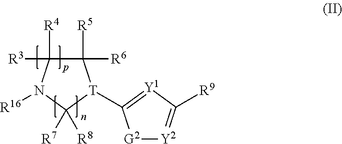

wherein R³, R⁴, R⁵, R⁶, R⁷, R⁸, R⁹, G², T, Y¹, Y², n and p are as defined for a compound of formula I and R¹⁶ is hydrogen, a protecting group, such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl. These compounds, including salts and N-oxides thereof, are useful as intermediates in the synthesis of compounds of formula I. Preferred definitions of R³, R⁴, R⁵, R⁶, R⁷, R⁸, R⁹, G², T, Y¹, Y², n and p are as defined for formula I.

The invention also includes compounds of formula Ill

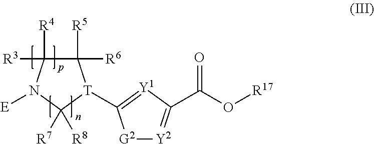

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M,

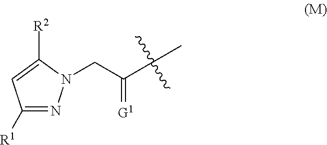

R¹, R², R³, R⁴, R⁵, R⁶, R⁷, R⁸, G¹, G², T, Y¹, Y², n and p are as defined for a compound of formula I and R¹⁷ is C₁-C₆alkyl. These compounds, including salts and N-oxides thereof, are useful as intermediates in the synthesis of compounds of formula I. Preferred definitions of R¹, R², R³, R⁴, R⁵, R⁶, R⁷, R⁸, G¹, G², T, Y¹, Y², n and p are as defined for formula I.

The invention also includes compounds of formula IV

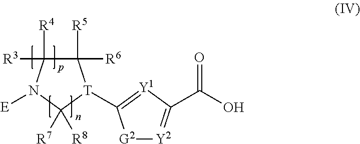

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M

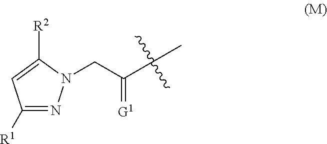

and R¹, R², R³, R⁴, R⁵, R⁶, R⁷, R⁸, G¹, G², T, Y¹, Y², n and p are as defined for a compound of formula I. These compounds, including salts and N-oxides thereof, are useful as intermediates in the synthesis of compounds of formula I. Preferred definitions of R¹, R², R³, R⁴, R⁵, R⁶, R⁷, R⁸, G¹, G², T, Y¹, Y², n and p are as defined for formula I.

The invention also includes compounds of formula V

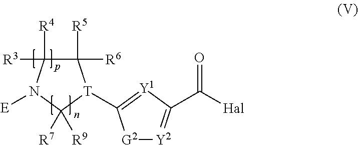

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M,

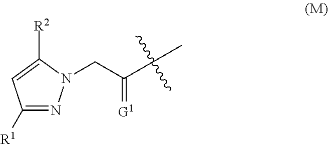

R¹, R², R³, R⁴, R⁵, R⁶, R⁷, R⁸, G¹, G², T, Y¹, Y², n and p are as defined for a compound of formula I and Hal is halogen, preferably chloro. These compounds, including salts and N-oxides thereof, are useful as intermediates in the synthesis of compounds of formula I. Preferred definitions of R¹, R², R³, R⁴, R⁵, R⁶, R⁷, R⁸, G¹, G², T, Y¹, Y², n and p are as defined for formula I.

The invention also includes compounds of formula VI

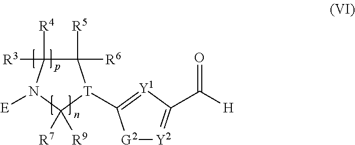

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M

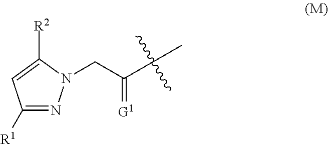

and R¹, R², R³, R⁴, R⁵, R⁶, R⁷, R⁸, G¹, G², T, Y¹, Y², n and p are as defined for a compound of formula I. These compounds, including salts and N-oxides thereof, are useful as intermediates in the synthesis of compounds of formula I. Preferred definitions of R¹, R², R³, R⁴, R⁵, R⁶, R⁷, R⁸, G¹, G², T, Y¹, Y², n and p are as defined for formula I.

The invention also includes compounds of formula VII

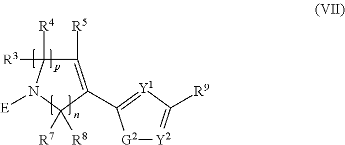

wherein E is hydrogen or a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; and R³, R⁴, R⁵, R⁷, R⁸, R⁹, G², Y¹, Y², n and p are as defined for a compound of formula I. These compounds, including salts and N-oxides thereof, are useful as intermediates in the synthesis of compounds of formula I. Preferred definitions of R³, R⁴, R⁵, R⁷, R⁸, R⁹, G², Y¹, Y², n and p are as defined for formula I.

Preferred individual compounds of formula I are:
4-[4-(2-chloro-6-fluoro-benzyloxycarbonyl)-thiazol-2-yl]-1-[2-(5-methyl-3-trifluoromethyl-pyrazol-1-yl)-acetyl]-piperidine-3-carboxylic acid methyl ester;
4-[4-(2-chloro-6-fluoro-benzyloxycarbonyl)-thiazol-2-yl]-1-[2-(5-methyl-3-trifluoromethyl-pyrazol-1-yl)-acetyl]-piperidine-2-carboxylic acid methyl ester;
1-[2-(3,5-bis-difluoromethyl-pyrazol-1-yl)-acetyl]-4-[4-(2-chloro-6-fluoro-benzyloxycarbonyl)-thiazol-2-yl]-piperidine-3-carboxylic acid methyl ester;
1-[2-(3,5-bis-difluoromethyl-pyrazol-1-yl)-acetyl]-4-[4-(2-chloro-6-fluoro-benzyloxycarbonyl)-thiazol-2-yl]-piperidine-2-carboxylic acid methyl ester;
4-[4-(2-chloro-6-fluoro-benzyloxycarbonyl)-thiazol-2-yl]-1-[2-(5-methyl-3-trifluoromethyl-pyrazol-1-yl)-acetyl]-piperazine-2-carboxylic acid methyl ester;
1-[4-(2-chloro-6-fluoro-benzyloxycarbonyl)-thiazol-2-yl]-4-[2-(5-methyl-3-trifluoromethyl-pyrazol-1-yl)-acetyl]-piperazine-2-carboxylic acid methyl ester;
4-[2-(3,5-bis-difluoromethyl-pyrazol-1-yl)-acetyl]-1-[4-(2-chloro-6-fluoro-benzyloxycarbonyl)-thiazol-2-yl]-piperazine-2-carboxylic acid methyl ester;
1-[2-(3,5-bis-difluoromethyl-pyrazol-1-yl)-acetyl]-4-[4-(2-chloro-6-fluoro-benzyloxycarbonyl)-thiazol-2-yl]-piperazine-2-carboxylic acid methyl ester;
4-{4-[methyl-(1,2,3,4-tetrahydro-naphthalen-1-yl)-carbamoyl]-thiazol-2-yl}-1-[2-(5-methyl-3-trifluoromethyl-pyrazol-1-yl)-acetyl]-piperidine-3-carboxylic acid methyl ester;
4-{4-[methyl-(1,2,3,4-tetrahydro-naphthalen-1-yl)-carbamoyl]-thiazol-2-yl}-1-[2-(5-methyl-3-trifluoromethyl-pyrazol-1-yl)-acetyl]-piperidine-2-carboxylic acid methyl ester;
1-[2-(3,5-bis-difluoromethyl-pyrazol-1-yl)-acetyl]-4-{4-[methyl-(1,2,3,4-tetrahydro-naphthalen 1-yl)-carbamoyl]-thiazol-2-yl}-piperidine-3-carboxylic acid methyl ester;
1-[2-(3,5-bis-difluoromethyl-pyrazol-1-yl)-acetyl]-4-[4-[-methyl-(1,2,3,4-tetrahydro-naphthalen-1-yl-carbamoyl)-thiazol-2-yl]-piperidine-2-carboxylic acid methyl ester;
1-{4-[methyl-(1,2,3,4-tetrahydro-naphthalen-1-yl)-carbamoyl]-thiazol-2-yl}-4-[2-(5-methyl-3-trifluoromethyl-pyrazol-1-yl)-acetyl]-piperazine-2-carboxylic acid methyl ester;
4-{4-[methyl-(1,2,3,4-tetrahydro-naphthalen-1-yl)-carbamoyl]-thiazol-2-yl}-1-[2-(5-methyl-3-trifluoromethyl-pyrazol-1-yl)-acetyl]-piperazine-2-carboxylic acid methyl ester;
1-[2-(3,5-bis-difluoromethyl-pyrazol-1-yl)-acetyl]-4-{4-[methyl-(1,2,3,4-tetrahydronaphthalen-1-yl)-carbamoyl]-thiazol-2-yl}-piperazine-2-carboxylic acid methyl ester;
4-[2-(3,5-bis-difluoromethyl-pyrazol-1-yl)-acetyl]-1-{4-[methyl-(1,2,3,4-tetrahydronaphthalen-1-yl)-carbamoyl]-thiazol-2-yl}-piperazine-2-carboxylic acid methyl ester;
4-{4-[5-(2,6-difluoro-phenyl)-4,5-dihydro-isoxazol-3-yl]-thiazol-2-yl}-1-[2-(5-methyl-3-trifluoromethyl-pyrazol-1-yl)-acetyl]-piperidine-3-carboxylic acid methyl ester;
4-{4-[5-(2,6-difluoro-phenyl)-4,5-dihydro-isoxazol-3-yi]-thiazol-2-yl}-1-[2-(5-methyl-3-trifluoromethyl-pyrazol-1-yl)-acetyl]-piperidine-2-carboxylic acid methyl ester;
1-[2-(3,5-bis-difluoromethyl-pyrazol-1-yl)-acetyl]-4-{4-[5-(2,6-difluoro-phenyl)-4,5-dihydro-isoxazol-3-yl]-thiazol-2-yl}-piperidine-3-carboxylic acid methyl ester;
1-[2-(3,5-bis-difluoromethyl-pyrazol-1-yl)-acetyl]-4-{4-[5-(2,6-difluoro-phenyl)-4,5-dihydro-isoxazol-3-yl]-thiazol-2-yl}-piperidine-2-carboxylic acid methyl ester;
1-{4-[5-(2,6-difluoro-phenyl)-4,5-dihydro-isoxazol-3-yl]-thiazol-2-yl}-4-[2-(5-methyl-3-trifluoromethyl-pyrazol-1-yl)-acetyl]-piperazine-2-carboxylic acid methyl ester;
4-{4-[5-(2,6-difluoro-phenyl)-4,5-dihydro-isoxazol-3-yl]-thiazol-2-yl}-1-[2-(5-methyl-3-trifluoromethyl-pyrazol-1-yl)-acetyl]-piperazine-2-carboxylic acid methyl ester;
1-[2-(3,5-bis-difluoromethyl-pyrazol-1-yl)-acetyl]-4-{4-[5-(2,6-difluoro-phenyl)-4,5-dihydro-isoxazol-3-yl]-thiazol-2-yl}-piperazine-2-carboxylic acid methyl ester; and
4-[2-(3,5-bis-diflu oromethyl-pyrazol-1-yl)-acetyl]-1-{4-[5-(2,6-difluoro-phenyl)-4,5-dihydro-isoxazol-3-yl]-thiazol-2-yl}-piperazine-2-carboxylic acid methyl ester.

Compounds of the present invention can be made as shown in the following schemes. Throughout this description, the group M, wherein R¹, R² and G¹ are as defined for formula I, stands for:

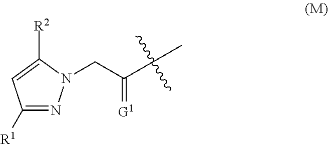

The compounds of formula IX, wherein R³, R⁴, R⁵, R⁶, R⁷, R⁸, R¹², G², T, Y¹, Y², n and p are as defined for formula I and E is hydrogen, a protecting group such as acetyl, benzyl or tert-butoxycarbonyl or a group M, can be obtained by transformation of a compound of formula V, wherein R³, R⁴, R⁵, R⁶, R⁷, R⁸, G², T, Y¹, Y², n and p are as defined for formula I, Hal is halogen, preferably chloro, and E is hydrogen, a protecting group such as acetyl, benzyl or tert-butoxycarbonyl or a group M, with a compound of formula VIII, wherein R¹² is as defined for formula I and a base, such as pyridine, triethylamine, ethyl diisopropylamine. This is shown in Scheme 1.

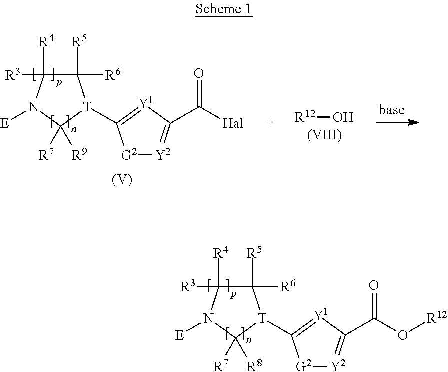

The compounds of formula V, wherein R³, R⁴, R⁵, R⁶, R⁷, R⁸, G², T, Y¹, Y², n and p are as defined for formula I, Hal is halogen, preferably chloro, and E is hydrogen, a protecting group such as acetyl, benzyl or tert-butoxycarbonyl or a group M, can be obtained by transformation of a compound of formula IV, wherein R³, R⁴, R⁵, R⁶, R⁷, Ra, G², T, Y¹, Y², n and p are as defined for formula I and E is hydrogen, a protecting group such as acetyl, benzyl or tert-butoxycarbonyl or a group M, with a thionyl halide, such as thionyl chloride, or a phosphoryl halide, such as phosphoryl chloride. This is shown in Scheme 2.

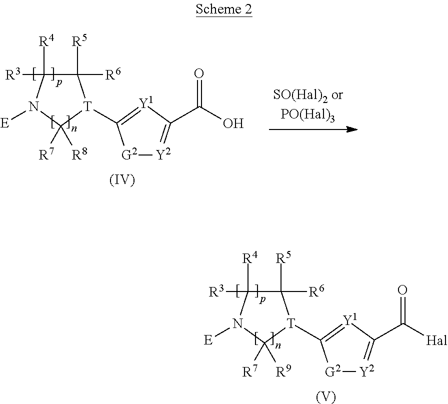

The compounds of formula IV, wherein R³, R⁴, R⁵, R⁶, R⁷, R⁸, G², T, Y¹, Y², n and p are as defined for formula I and E is hydrogen, a protecting group such as acetyl, benzyl or tert-butoxycarbonyl or a group M, can be obtained by transformation of a compound of formula Ill, wherein R³, R⁴, R⁵, R⁶, R⁷, R⁸, G², T, Y¹, Y², n and p are as defined for formula I, R¹⁷ is C₁-C₆alkyl and E is hydrogen, a protecting group such as acetyl, benzyl or tert-butoxycarbonyl or a group M, with a base, such as lithium hydroxide, sodium hydroxide, sodium carbonate, potassium hydroxide or potassium carbonate. This is shown in Scheme 3.

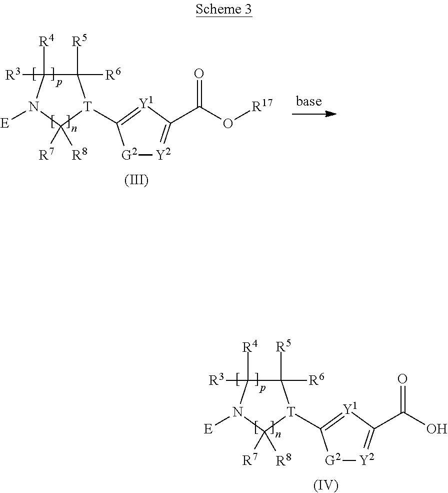

Alternatively, the compounds of formula IX, wherein R³, R⁴, R⁵, R⁶, R⁷, R⁸, R¹², G², T, Y¹, Y², n and p are as defined for formula I and E is hydrogen, a protecting group such as acetyl, benzyl or tert-butoxycarbonyl or a group M, can be obtained by transformation of a compound of formula IV, wherein R³, R⁴, R⁵, R⁶, R⁷, R⁸, G², T, Y¹, Y², n and p are as defined for formula I and E is hydrogen, a protecting group such as acetyl, benzyl or tert-butoxycarbonyl or a group M, with a compound of formula VIII, wherein R¹² is as defined for formula I and an acid, such as hydrochloric acid or sulphuric acid. This is shown in Scheme 4.

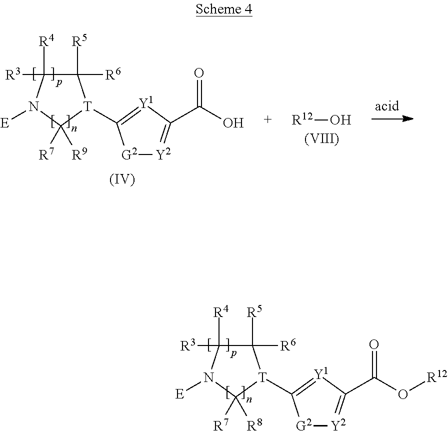

The compounds of formula XI, wherein R³, R⁴, R⁵, R⁶, R⁷, R⁸, R¹², R¹³, G², T, Y¹, Y², n and p are as defined for formula I and E is hydrogen, a protecting group such as acetyl, benzyl or tert-butoxycarbonyl or a group M, can be obtained by transformation of a compound of formula V, wherein R³, R⁴, R⁵, R⁶, R⁷, R⁸, G², T, Y¹, Y², n and p are as defined for formula I, Hal is halogen, preferably chloro, and E is hydrogen, a protecting group such as acetyl, benzyl or tert-butoxycarbonyl or a group M, with a compound of formula X, wherein R¹² and R¹³ are as defined for formula I and a base, such as pyridine, triethylamine, ethyl diisopropylamine. This is shown in Scheme 5.

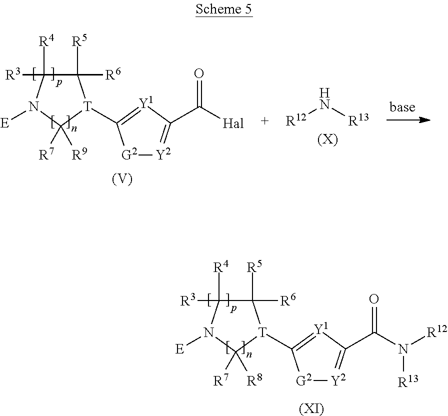

Alternatively, the compounds of formula XI, wherein R³, R⁴, R⁵, R⁶, R⁷, R⁸, R¹², R¹³, G², T, Y¹, Y², n and p are as defined for formula I and E is hydrogen, a protecting group such as acetyl, benzyl or tert-butoxycarbonyl or a group M, can be obtained by transformation of a compound of formula IV, wherein R³, R⁴, R⁵, R⁶, R⁷, R⁸, G², T, Y¹, Y², n and p are as defined for formula I and E is hydrogen, a protecting group such as acetyl, benzyl or tert-butoxycarbonyl or a group M, with a compound of formula X, wherein R¹² and R¹³ are as defined for formula I, an activating reagent, such as BOP, PyBOP, EDCI, CDI or HATU, and a base, such as pyridine, triethylamine, ethyl diisopropylamine. This is shown in Scheme 6.

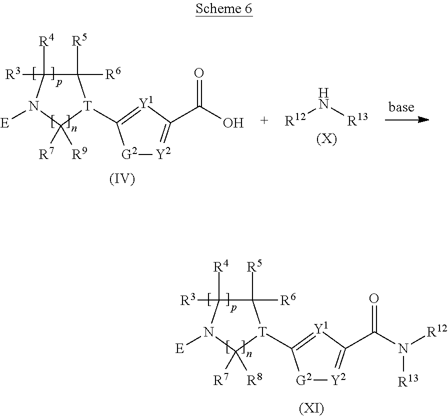

The compounds of formula XIII, wherein R³, R⁴, R⁵, R⁶, R⁷, R⁸, R¹⁴, G², T, Y¹, Y², n and p are as defined for formula I and E is hydrogen, a protecting group such as acetyl, benzyl or tert-butoxycarbonyl or a group M, can be obtained by transformation of a compound of formula VI, wherein R³, R⁴, R⁵, R⁶, R⁷, R⁸, G², T, Y¹, Y², n and p are as defined for formula I and E is hydrogen, a protecting group such as acetyl, benzyl or tert-butoxycarbonyl or a group M, with a compound of formula XII, wherein R¹⁴ is as defined for formula, and hydroxylamine and sodium hypochlorite. This is shown in Scheme 7.

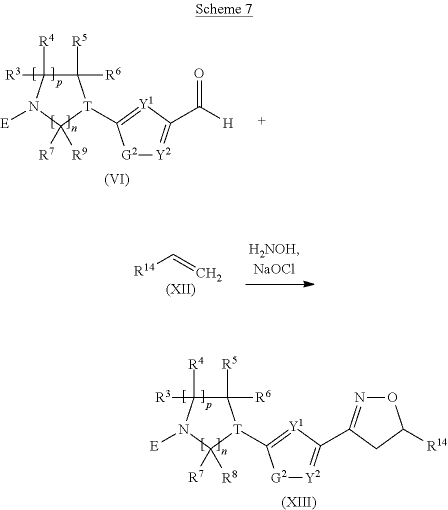

The compounds of formula XIV, wherein R³, R⁴, R⁵, R⁶, R⁷, R⁸, R⁹, G², T, Y¹, Y², n and p are as defined for formula I and E is hydrogen, a protecting group such as acetyl, benzyl or tert-butoxycarbonyl, can be obtained by transformation of a compound of formula VII, wherein R³, R⁴, R⁵, R⁷, R⁸, R⁹, G², Y¹, Y², n and p are as defined for formula I and E is hydrogen or a protecting group such as acetyl, benzyl or tert-butoxycarbonyl, with hydrogen and a catalyst, such as palladium on charcoal or Raney-nickel, or with a hydride, such as lithium aluminium hydride. This is shown in Scheme 8.

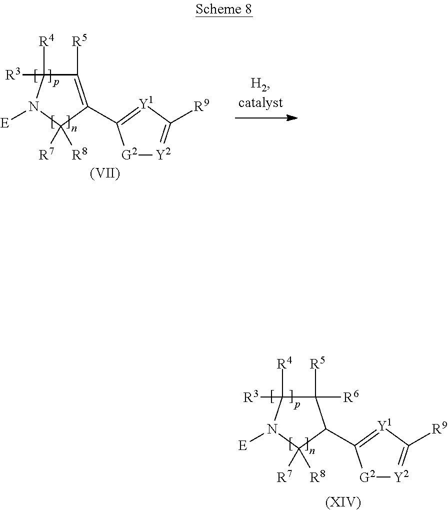

The compounds of formula VII, wherein R³, R⁴, R⁵, R⁷, R⁸, R⁹, G², Y¹, Y², n and p are as defined for formula I and E is hydrogen or a protecting group such as acetyl, benzyl or tert-butoxycarbonyl, can be obtained by transformation of a compound of formula XV, wherein R³, R⁴, R⁵, R⁷, R⁸, n and p are as defined for formula I, R¹⁸ and R¹⁹ each independently are hydroxyl or C₁₋₄alkoxy or together with the interjacent boron atom form a five- or six-membered saturated heterocyclic ring, and E is hydrogen or a protecting group such as acetyl, benzyl or tert-butoxycarbonyl, with a compound of formula XVI, wherein R⁹, G², Y¹, Y² and Hal is halogen, preferably bromo or iodo, and a transition metal catalyst, such as tetrakis(triphenylphosphine)palladium, and a ligand. This is shown in Scheme 9.

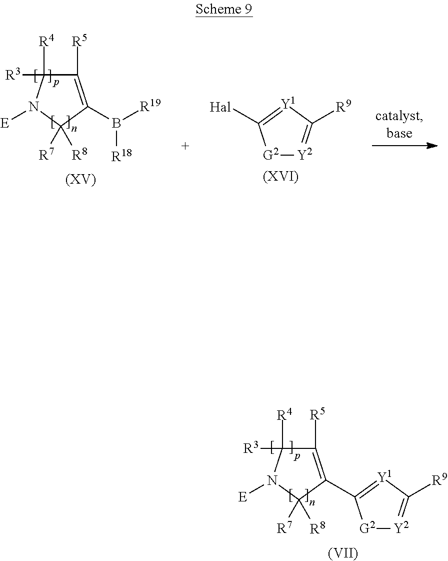

The compounds of formula XVIII, wherein R³, R⁴, R⁵, R⁶, R⁷, R⁸, R⁹, G², Y¹, Y², n and p are as defined for formula I and E is hydrogen, a protecting group such as acetyl, benzyl or tert-butoxycarbonyl or a group M, can be obtained by transformation of a compound of formula XVII, wherein R³, R⁴, R⁵, R⁶, R⁷, R⁸, n and p are as defined for formula I and E is hydrogen, a protecting group such as acetyl, benzyl or tert-butoxycarbonyl or a group M, with a compound of formula XVI, wherein R⁹, G², Y¹, Y² and Hal is halogen, preferably chloro, bromo or iodo, with a transition metal catalyst and a ligand or with a base. This is shown in Scheme 10.

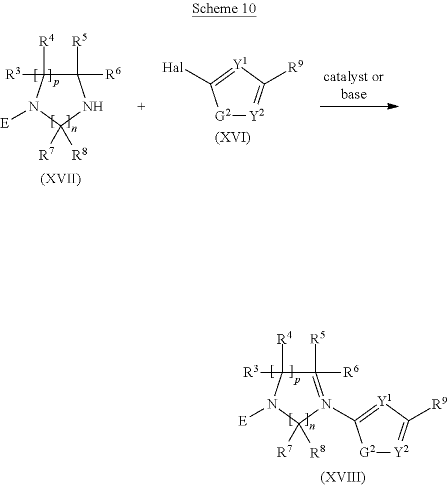

Surprisingly, it has now been found that the novel compounds of formula I have, for practical purposes, a very advantageous level of biological activity for protecting plants against diseases that are caused by fungi.

The compounds of formula I can be used in the agricultural sector and related fields of use e.g. as active ingredients for controlling plant pests or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and may be used for protecting numerous cultivated plants. The compounds of formula I can be used to inhibit or destroy the pests that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula I as dressing agents for the treatment of plant propagation material, e.g., seed, such as fruits, tubers or grains, or plant cuttings (for example rice), for the protection against fungal infections as well as against phytopathogenic fungi occurring in the soil. The propagation material can be treated with a composition comprising a compound of formula I before planting: seed, for example, can be dressed before being sown. The active ingredients according to the invention can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore the compounds according to present invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula I are, for example, effective against the phytopathogenic fungi of the following classes: Fungi imperfecti (e.g. Alternaria spp.), Basidiomycetes (e.g. Corticium spp., Ceratobasidium spp., Waitea spp., Thanatephorus spp., Rhizoctonia spp., Hemileia spp., Puccinia spp., Phakopsora spp., Ustilago spp., Tilletia spp.), Ascomycetes (e.g. Venturia spp., Blumeria spp., Erysiphe spp., Podosphaera spp., Uncinula spp., Monilinia spp., Sclerotinia spp., Colletotrichum spp., Glomerella spp., Fusarium spp., Gibberella spp., Monographella spp., Phaeosphaeria spp., Mycosphaerella spp., Cercospora spp., Pyrenophora spp., Rhynchosporium spp., Magnaporthe spp., Gaeumannomyces spp., Oculimacula spp., Ramularia spp., Botryotinia spp.) and Oomycetes (e.g. Phytophthora spp., Pythium spp., Plasmopara spp., Peronospora spp., Pseudoperonospora spp. Bremia spp).

Outstanding activity is observed against downy mildew (e.g. Plasmopara viticola) and late blight (e.g. Phytophthora infestans). Furthermore, the novel compounds of formula I are effective against phytopathogenic gram negative and gram positive bacteria (e.g. Xanthomonas spp, Pseudomonas spp, Erwinia amylovora, Ralstonia spp.) and viruses (e.g. tobacco mosaic virus).

Within the scope of present invention, target crops and/or useful plants to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cihnamomum, camphor) or plants such as tobacco, nuts, coffee, eggplants, sugar cane, tea, pepper, vines, hops, bananas and natural rubber plants, as well as turf and ornamentals.

The useful plants and/or target crops in accordance with the invention include conventional as well as genetically enhanced or engineered varieties such as, for example, insect resistant (e.g. Bt. and VIP varieties) as well as disease resistant, herbicide tolerant (e.g. glyphosate- and glufosinate-resistant maize varieties commercially available under the trade names RoundupReady® and LibertyLink®) and nematode tolerant varieties. By way of example, suitable genetically enhanced or engineered crop varieties include the Stoneville 5599BR cotton and Stoneville 4892BR cotton varieties.

The term “useful plants” and/or “target crops” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” and/or “target crops” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

The term “useful plants” and/or “target crops” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The term “locus” of a plant as used herein is intended to embrace the place on which the plants are growing, where the plant propagation materials of the plants are sown or where the plant propagation materials of the plants will be placed into the soil. An example for such a locus is a field, on which crop plants are growing.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The compounds of formula I may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula I are normally used in the form of compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula I may be used in the form of (fungicidal) compositions for controlling or protecting against phytopathogenic microorganisms, comprising as active ingredient at least one compound of formula I or of at least one preferred individual compound as above-defined, in free form or in agrochemically usable salt form, and at least one of the above-mentioned adjuvants.

The invention provides a composition, preferably a fungicidal composition, comprising at least one compound formula I an agriculturally acceptable carrier and optionally an adjuvant. An agricultural acceptable carrier is for example a carrier that is suitable for agricultural use. Agricultural carriers are well known in the art. Preferably said composition may comprise at least one or more pesticidally active compounds, for example an additional fungicidal active ingredient in addition to the compound of formula I.

The compound of formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities. Examples of suitable additional active ingredients include the following: Azoxystrobin (131860-33-8), Dimoxystrobin (149961-52-4), Enestrobin (238410-11-2), Fluoxastrobin (193740-76-0), Kresoxim-methyl (143390-89-0), Metominostrobin (133408-50-1), Orysastrobin (248593-16-0), Picoxystrobin (117428-22-5), Pyraclostrobin (175013-18-0), trifloxystrobin (141517-21-7), Azaconazole (60207-31-0), Bromuconazole (116255-48-2), Cyproconazole (94361-06-5), Difenoconazole (119446-68-3), Diniconazole (83657-24-3), Diniconazole-M (83657-18-5), Epoxiconazole (13385-98-8), Fenbuconazole (114369-43-6), Fluquinconazole (136426-54-5), Flusilazole (85509-19-9), Flutriafol (76674-21-0), Hexaconazole (79983-71-4), Imazalil (58594-72-2), Imibenconazole (86598-92-7), Ipconazole (125225-28-7), Metconazole (125116-23-6), Myclobutanil (88671-89-0), Oxpoconazole (174212-12-5), Pefurazoate (58011-68-0), Penconazole (66246-88-6), Prochloraz (67747-09-5), Propiconazole (60207-90-1), Prothioconazole (178928-70-6), Simeconazole (149508-90-7), Tebuconazole (107534-96-3), Tetraconazole (112281-77-3), Triadimefon (43121-43-3), Triadimenol (55219-65-3), Triflumizole (99387-89-0), Triticonazole (131983-72-7), Diclobutrazol (76738-62-0), Etaconazole (60207-93-4), Fluconazole (86386-73-4), Fluconazole-cis (112839-32-4), Thiabendazole (148-79-8), Quinconazole (103970-75-8), Fenpiclonil (74738-17-3), Fludioxonil (131341-86-1), Cyprodinil (121552-61-2), Mepanipyrim (110235-47-7), Pyrimethanil (53112-28-0), Aldimorph (91315-15-0), Dodemorph (1593-77-7), Fenpropimorph (67564-91-4), Tridemorph (81412-43-3), Fenpropidin (67306-00-7), Spiroxamine (118134-30-8), Isopyrazam (881685-58-1), Sedaxane (874967-67-6), Bixafen (581809-46-3), Penthiopyrad (183675-82-3), Fluxapyroxad (907204-31-3), Boscalid (188425-85-6), Penflufen (494793-67-8), Fluopyram (658066-35-4), Mandipropamid (374726-62-2), Benthiavalicarb (413615-35-7), Dimethomorph (110488-70-5), Chlorothalonil (1897-45-6), Fluazinam (79622-59-6), Dithianon (3347-22-6), Metrafenone (220899-03-6), Tricyclazole (41814-78-2), Mefenoxam (70630-17-0), Metalaxyl (57837-19-1), Acibenzolar (126448-41-7) (Acibenzolar-S-methyl (126448-41-7)), Mancozeb (8018-01-7), Ametoctradine (865318-97-4) Cyflufenamid (180409-60-3), and Kresoxim-methyl (143390-89-0), Ipconazole (125225-28-7), Amisulbrom (348635-87-0), Cyflufenamid (180409-60-3), Ethaboxam (16650-77-3), Fluopicolide (239110-15-7), Fluthianil (304900-25-2), Isotianil (224049-04-1), Proquinazid (189278-12-4), Valiphenal (283159-90-0), 1-methyl-cyclopropene (3100-04-7), Trifloxystrobin (141517-21-7), Sulfur (7704-34-9), Copper ammoniumcarbonate (CAS 33113-08-5); Copper oleate (CAS 1120-44-1); Folpet (133-07-3), Quinoxyfen (124495-18-7), Captan (133-06-2), Fenhexamid (126833-17-8), Glufosinate and its salts (51276-47-2, 35597-44-5 (S-isomer)), Glyphosate (1071-83-6) and its salts (69254-40-6 (Diammonium), 34494-04-7 (Dimethylammonium), 38641-94-0 (Isopropylammonium), 40465-66-5 (Monoammonium), 70901-20-1 (Potassium), 70393-85-0 (Sesquisodium), 81591-81-3 (Trimesium)), 1-methyl-3-difluoromethyl-1H-pyrazole-4-carboxylic acid (2-dichloromethylene-3-ethyl-1-methyl-indan-4-yl)-amide (1072957-71-1), 1-methyl-3-difluoromethyl-1H-pyrazole-4-carboxylic acid (4′-methylsulfanyl-biphenyl-2-yl)-amide, 1-methyl-3-difluoromethyl-4H-pyrazole-4-carboxylic acid [2-(2,4-dichloro-phenyl)-2-methoxy-1-methyl-ethyl]-amide, (5-Chloro-2,4-dimethyl-pyridin-3-yl)-(2,3,4-trimethoxy-6-methyl-phenyl)-methanone, (5-Bromo-4-chloro-2-methoxy-pyridin-3-yl)-(2,3,4-trimethoxy-6-methyl-phenyl)-methanone, 2-{2-[(E)-3-(2,6-Dichloro-phenyl)-1-methyl-prop-2-en-(E)-ylideneaminooxymethyl]-phenyl}-2-[(Z)-methoxyimino]-N-methyl-acetamide, 3-[5-(4-Chloro-phenyl)-2,3-dimethyl-isoxazolidin-3-yl]-pyridine.

Another aspect of invention is related to the use of a compound of formula I or of a preferred individual compound as above-defined, of a composition comprising at least one compound of formula I or at least one preferred individual compound as above-defined, or of a fungicidal mixture comprising at least one compound of formula I or at least one preferred individual compound as above-defined, in admixture with other fungicides, as described above, for controlling or preventing infestation of plants, e.g. useful plants such as crop plants, propagation material thereof, e.g. seeds, harvested crops, e.g. harvested food crops, or non-living materials by phytopathogenic microorganisms, preferably fungal organisms.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a compound of formula I or of a preferred individual compound as above-defined as active ingredient to the plants, to parts of the plants or to the locus thereof, to the propagation material thereof, or to any part of the non-living materials.

Controlling or preventing means reducing infestation by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, which comprises the application of a compound of formula I, or an agrochemical composition which contains at least one of said compounds, is foliar application. The frequency of application and the rate of application will depend on the risk of infestation by the corresponding pathogen. However, the compounds of formula I can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation, e.g. a composition containing the compound of formula I, and, if desired, a solid or liquid adjuvant or monomers for encapsulating the compound of formula I, may be prepared in a known manner, typically by intimately mixing and/or grinding the compound with extenders, for example solvents, solid carriers and, optionally, surface active compounds (surfactants).

The agrochemical formulations and/or compositions will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

Whereas it is preferred to formulate commercial products as concentrates, the end user will normally use dilute formulations.

The following non-limiting examples illustrate the above-described invention in more detail.

### EXAMPLE 1

### This example illustrates the preparation of 4-{4-[5-(2,6-difluoro-phenyl)-4,5-dihydro-isoxazol-3-yl]-thiazol-2-yl}-1-[2-(5-methyl-3-trifluoromethyl-pyrazol-1-yl)-acetyl]-piperazine-2-carboxylic acid methyl ester (compound I.az.038)

### a) Preparation of 3-(2-bromo-thiazol-4-yl)-5-(2,6-difluoro-phenyl)-4,5-dihydro-isoxazole

5.0 ml of a 50% aqueous solution of hydroxylamine were added to a suspension of 2-bromo-4-formylthiazole (4.0 g, 20 mmol) in 50 ml of ethanol. The reaction mixture was heated to 60 OC for 2 h. The resulting solution was cooled to room temperature and diluted with 80 ml of tetrahydrofuran. 2,6-difluorostyrene (4.3 g, 30 mmol) was added to the reaction mixture, followed by the dropwise addition of 98 ml of a 15% aqueous solution of sodium hypochlorite. The reaction mixture was stirred for 16 h at room temperature and subsequently quenched by addition of water. The phases were separated and the aqueous phase was extracted with ethyl acetate. The combined organic layer was washed with 5% aqueous sodium bicarbonate solution and brine, dried over sodium sulfate and concentrated under reduced pressure. The remainder was purified by column chromatography on silica gel (cyclohexane/ethyl acetate 4:1) to obtain 3-(2-bromo-thiazol-4-yl)-5-(2,6-difluoro-phenyl)-4,5-dihydro-isoxazole as light yellow cristalls. M.p. 135-142° C. ¹H-NMR (400 MHz, CDCl₃): δ=3.63 (dd, 1H), 3.81 (dd, 1H), 6.09 (dd, 1H), 6.92 (t, 2H), 7.28-7.35 (m, 1H), 7.78 (s, 1H). MS: m/z=347 (M+2).

### b) Preparation of 4-{4-[5-(2,6-difluoro-phenyl)-4,5-dihydro-isoxazol-3-yl]-thiazol-2-yl}-piperazine-1,2-dicarboxylic acid 1-tert-butyl ester 2-methyl ester

Potassium carbonate (1.2 g, 8.7 mmol) was added to a solution of 3-(2-bromo-thiazol-4-yl)-5-(2,6-difluoro-phenyl)-4,5-dihydro-isoxazole (1.25 g, 3.6 mmol) and 1-N-Boc-2-piperazinecarboxylic acid methyl ester (1.1 g, 4.4 mmol) in 15 ml of acetonitrile. The reaction mixture was stirred for 150 h at 90° C. and subsequently cooled and filtered. The solid potassium salt was washed with ethyl acetate and the filtrate as well as the washing layers were combined and evaporated. The remainder was purified by column chromatography on silica gel (cyclohexane/ethyl acetate 4:1) to obtain 4-{4-[5-(2,6-difluoro-phenyl)-4,5-dihydro-isoxazol-3-yl]-thiazol-2-yl}-piperazine-1,2-dicarboxylic acid 1-tert-butyl ester 2-methyl ester as a light yellow foam. M.p. 68-72° C. ¹H-NMR (400 MHz, CDCl₃): δ=1.42-1.53 (m, 9H), 3.09 (dd, 1H), 3.33 (dt, 2H), 3.52 (dd, 1H), 3.69 (t, 1H), 3.74 (s, 3H), 3.91-4.08 (m, 2H), 4.42 (dd, 1H), 4.70-4.93 (m, 1H), 6.02 (t, 1H), 6.89 (t, 2H), 7.00 (s, 1H), 7.24-7.31 (m, 1H). MS: m/z=509 (M+1).

### c) Preparation of 4-{4-[5-(2,6-difluoro-phenyl)-4,5-dihydro-isoxazol-3-yl]-thiazol-2-yl}-piperazine-2-carboxylic acid methyl ester hydrochloride

12.5 ml of a 1.25 M solution of hydrochloric acid in ethanol were added to a solution of 4-{4-[5-(2,6-difluoro-phenyl)-4,5-dihydro-isoxazol-3-yl]-thiazol-2-yl}-piperazine-1,2-dicarboxylic acid 1-tert-butyl ester 2-methyl ester (0.8 g, 1.6 mmol) in 13 ml of a 1:1 mixture of dichloromethane and methanol. This reaction mixture was stirred for 16 h at room temperature and then evaporated to directly deliver 4-{4-[5-(2,6-difluoro-phenyl)-4,5-dihydro-isoxazol-3-yl]-thiazol-2-yl}-piperazine-2-carboxylic acid methyl ester hydrochloride as a light orange foam. ¹H-NMR (400 MHz, CDCl₃): δ=3.21 (dd, 1H), 3.39-3.48 (m, 2H), 3.59 (dd, 1H), 3.68 (t, 1H), 3.77-3.86 (m, 5H), 4.25 (dd, 1H), 4.51-4.57 (m, 1H), 5.99 (t, 1H), 6.32 (bs, 1H), 7.16 (t, 2H), 7.42 (s, 1H), 7.50 (q, 1H). MS: m/z=409 (M+1-HCl).

### d) Preparation of 4-{4-[5-(2,6-difluoro-phenyl)-4,5-dihydro-isoxazol-3-yl]-thiazol-2-yl}-1-[2-(5-methyl-3-trifluoromethyl-pyrazol-1-yl)-acetyl]-piperazine-2-carboxylic acid methyl ester (compound I.az.038)

To a solution of 4-{4-[5-(2,6-difluoro-phenyl)-4,5-dihydro-isoxazol-3-yl]-thiazol-2-yl}-piperazine-2-carboxylic acid methyl ester hydrochloride (0.5 g, 1.0 mmol) in 4 ml of N,N-dimethylformamide were added at 0° C. sequentially ethyl(diisopropyl)amine (0.3 g, 2.5 mmol), (5-methyl-3-trifluoromethyl-pyrazol-1-yl)-acetic acid (0.2 g, 1.1 mmol) and benzotriazol-1-yloxy-tris(dimethylamino)phosphonium hexafluorophosphate (0.5 g, 1.1 mmol). The reaction mixture was stirred for 16 h at room temperature, then poured on water and extracted with ethyl acetate. The combined organic layer was washed with saturated aqueous sodium bicarbonate solution and brine, dried over sodium sulfate and evaporated. The residue is purified by column chromatography on silica gel (cyclohexane/ethyl acetate 4:1) to give 4-{4-[5-(2,6-difluoro-phenyl)-4,5-dihydro-isoxazol-3-yl]-thiazol-2-yl}-1-[2-(5-methyl-3-trifluoromethyl-pyrazol-1-yl)-acetyl]-piperazine-2-carboxylic acid methyl ester (compound I.az.038) as light yellow foam. ¹H-NMR (400 MHz, CDCl₃): 8=2.31 (s, 3H), 3.12 (t, 1H), 3.34 (dd, 1H), 3.53 (dd, 1H), 3.64-3.76 (m, 4H), 3.89 (dd, 1H), 4.20 (dt, 1H), 4.44 (dd, 1H), 4.97 (dd, 1H), 5.22 (dd, 1H), 6.02 (t, 1H), 6.33 (d, 1H), 6.91 (t, 2H), 7.02 (s, 1H), 7.29 (q, 1H). MS: m/z=599 (M+1).

### EXAMPLE 2

### This example illustrates the preparation of 4-{4-[5-(2,6-difluoro-phenyl)-4,5-dihydro-isoxazol-3-yl]-thiazol-2-yl}-1-[2-(5-methyl-3-trifluoromethyl-pyrazol-1-yl)-acetyl]-piperazine-2-carboxylic acid (compound I.az.037)

### a) Preparation of 4-{4-[5-(2,6-difluoro-phenyl)-4,5-dihydro-isoxazol-3-yl]-thiazol-2-yl}-1-[2-(5-methyl-3-trifluoromethyl-pyrazol-1-yl)-acetyl]-piperazine-2-carboxylic acid (compound I.az.037)

To a solution of 4-{4-[5-(2,6-difluoro-phenyl)-4,5-dihydro-isoxazol-3-yl]-thiazol-2-yl}-1-[2-(5-methyl-3-trifluoromethyl-pyrazol-1-yl)-acetyl]-piperazine-2-carboxylic acid methyl ester (compound I.az.038, 0.3 g, 0.5 mmol) in 5 ml of tetrahydrofurane was added 0.8 ml of a 1 N sodium hydroxide solution. The reaction mixture was stirred for 2 h at room temperature and then concentrated under reduced pressure. The residue was taken up in water and washed with ethyl acetate. The aqueous phase was acidified with citric acid to pH 3 to 4 and then extracted with ethyl acetate. The organic layer was washed with water and brine, dried over sodium sulfate and evaporated to directly deliver 4-{4-[5-(2,6-difluoro-phenyl)-4,5-dihydro-isoxazol-3-yl]-thiazol-2-yl}-1-[2-(5-methyl-3-trifluoromethyl-pyrazol-1-yl)-acetyl]-piperazine-2-carboxylic acid (compound I.az.037) as white cristalls. M.p. 178-185° C. ¹H-NMR (400 MHz, CDCl₃): δ=2.27 (s, 3H), 3.04-3.20 (m, 2H), 3.33 (dd, 1H), 3.50 (dd, 1H), 3.62-3.71 (m, 2H), 3.89 (dd, 1H), 4.17 (dt, 1H), 4.45 (dd, 1H), 5.04-5.28 (m, 3H), 6.00 (t, 1H), 6.31 (d, 1H), 6.89 (t, 2H), 6.96 (s, 1H), 7.28 (q, 1H). MS: m/z=585 (M+1).

Table 1 below illustrates examples of individual compounds of formula I according to the invention.

TABLE 1
individual compounds of formula I according to the invention

| Comp. | | | | | | | | |
|---|---|---|---|---|---|---|---|---|
| No. | R¹ | R² | R³ | R⁵ | T | Y¹ | G² | Y² |
| 001 | F₃C | H₃C | CO₂H | H | CH | N | S | CH |
| 002 | F₃C | H₃C | CO₂CH₃ | H | CH | N | S | CH |
| 003 | F₃C | H₃C | CO₂CH₂CH₃ | H | CH | N | S | CH |
| 004 | F₃C | H₃C | CONH₂ | H | CH | N | S | CH |
| 005 | F₃C | H₃C | CONHCH₃ | H | CH | N | S | CH |
| 006 | F₃C | H₃C | CON(CH₃)₂ | H | CH | N | S | CH |
| 007 | F₃C | H₃C | H | CO₂H | CH | N | S | CH |
| 008 | F₃C | H₃C | H | CO₂CH₃ | CH | N | S | CH |
| 009 | F₃C | H₃C | H | CO₂CH₂CH₃ | CH | N | S | CH |
| 010 | F₃C | H₃C | H | CONH₂ | CH | N | S | CH |
| 011 | F₃C | H₃C | H | CONHCH₃ | CH | N | S | CH |
| 012 | F₃C | H₃C | H | CON(CH₃)₂ | CH | N | S | CH |
| 013 | F₃C | H₃C | CO₂H | H | CH | N | S | N |
| 014 | F₃C | H₃C | CO₂CH₃ | H | CH | N | S | N |
| 015 | F₃C | H₃C | CO₂CH₂CH₃ | H | CH | N | S | N |
| 016 | F₃C | H₃C | CONH₂ | H | CH | N | S | N |
| 017 | F₃C | H₃C | CONHCH₃ | H | CH | N | S | N |
| 018 | F₃C | H₃C | CON(CH₃)₂ | H | CH | N | S | N |
| 019 | F₃C | H₃C | H | CO₂H | CH | N | S | N |
| 020 | F₃C | H₃C | H | CO₂CH₃ | CH | N | S | N |
| 021 | F₃C | H₃C | H | CO₂CH₂CH₃ | CH | N | S | N |
| 022 | F₃C | H₃C | H | CONH₂ | CH | N | S | N |
| 023 | F₃C | H₃C | H | CONHCH₃ | CH | N | S | N |
| 024 | F₃C | H₃C | H | CON(CH₃)₂ | CH | N | S | N |
| 025 | F₃C | H₃C | CO₂H | H | CH | N | O | CH |
| 026 | F₃C | H₃C | CO₂CH₃ | H | CH | N | O | CH |
| 027 | F₃C | H₃C | CO₂CH₂CH₃ | H | CH | N | O | CH |
| 028 | F₃C | H₃C | CONH₂ | H | CH | N | O | CH |
| 029 | F₃C | H₃C | CONHCH₃ | H | CH | N | O | CH |
| 030 | F₃C | H₃C | CON(CH₃)₂ | H | CH | N | O | CH |
| 031 | F₃C | H₃C | H | CO₂H | CH | N | O | CH |
| 032 | F₃C | H₃C | H | CO₂CH₃ | CH | N | O | CH |
| 033 | F₃C | H₃C | H | CO₂CH₂CH₃ | CH | N | O | CH |
| 034 | F₃C | H₃C | H | CONH₂ | CH | N | O | CH |
| 035 | F₃C | H₃C | H | CONHCH₃ | CH | N | O | CH |
| 036 | F₃C | H₃C | H | CON(CH₃)₂ | CH | N | O | CH |
| 037 | F₃C | H₃C | CO₂H | H | N | N | S | CH |
| 038 | F₃C | H₃C | CO₂CH₃ | H | N | N | S | CH |
| 039 | F₃C | H₃C | CO₂CH₂CH₃ | H | N | N | S | CH |
| 040 | F₃C | H₃C | CONH₂ | H | N | N | S | CH |
| 041 | F₃C | H₃C | CONHCH₃ | H | N | N | S | CH |
| 042 | F₃C | H₃C | CON(CH₃)₂ | H | N | N | S | CH |
| 043 | F₃C | H₃C | H | CO₂H | N | N | S | CH |
| 044 | F₃C | H₃C | H | CO₂CH₃ | N | N | S | CH |
| 045 | F₃C | H₃C | H | CO₂CH₂CH₃ | N | N | S | CH |
| 046 | F₃C | H₃C | H | CONH₂ | N | N | S | CH |
| 047 | F₃C | H₃C | H | CONHCH₃ | N | N | S | CH |
| 048 | F₃C | H₃C | H | CON(CH₃)₂ | N | N | S | CH |
| 049 | F₃C | H₃C | CO₂H | H | N | N | S | N |
| 050 | F₃C | H₃C | CO₂CH₃ | H | N | N | S | N |
| 051 | F₃C | H₃C | CO₂CH₂CH₃ | H | N | N | S | N |
| 052 | F₃C | H₃C | CONH₂ | H | N | N | S | N |
| 053 | F₃C | H₃C | CONHCH₃ | H | N | N | S | N |
| 054 | F₃C | H₃C | CON(CH₃)₂ | H | N | N | S | N |
| 055 | F₃C | H₃C | H | CO₂H | N | N | S | N |
| 056 | F₃C | H₃C | H | CO₂CH₃ | N | N | S | N |
| 057 | F₃C | H₃C | H | CO₂CH₂CH₃ | N | N | S | N |
| 058 | F₃C | H₃C | H | CONH₂ | N | N | S | N |
| 059 | F₃C | H₃C | H | CONHCH₃ | N | N | S | N |
| 060 | F₃C | H₃C | H | CON(CH₃)₂ | N | N | S | N |
| 061 | F₃C | H₃C | CO₂H | H | N | N | O | CH |
| 062 | F₃C | H₃C | CO₂CH₃ | H | N | N | O | CH |
| 063 | F₃C | H₃C | CO₂CH₂CH₃ | H | N | N | O | CH |
| 064 | F₃C | H₃C | CONH₂ | H | N | N | O | CH |
| 065 | F₃C | H₃C | CONHCH₃ | H | N | N | O | CH |
| 066 | F₃C | H₃C | CON(CH₃)₂ | H | N | N | O | CH |
| 067 | F₃C | H₃C | H | CO₂H | N | N | O | CH |
| 068 | F₃C | H₃C | H | CO₂CH₃ | N | N | O | CH |
| 069 | F₃C | H₃C | H | CO₂CH₂CH₃ | N | N | O | CH |
| 070 | F₃C | H₃C | H | CONH₂ | N | N | O | CH |
| 071 | F₃C | H₃C | H | CONHCH₃ | N | N | O | CH |
| 072 | F₃C | H₃C | H | CON(CH₃)₂ | N | N | O | CH |
| 073 | H₃C | H₃C | CO₂H | H | CH | N | S | CH |
| 074 | H₃C | H₃C | CO₂CH₃ | H | CH | N | S | CH |
| 075 | H₃C | H₃C | CO₂CH₂CH₃ | H | CH | N | S | CH |
| 076 | H₃C | H₃C | CONH₂ | H | CH | N | S | CH |
| 077 | H₃C | H₃C | CONHCH₃ | H | CH | N | S | CH |
| 078 | H₃C | H₃C | CON(CH₃)₂ | H | CH | N | S | CH |
| 079 | H₃C | H₃C | H | CO₂H | CH | N | S | CH |
| 080 | H₃C | H₃C | H | CO₂CH₃ | CH | N | S | CH |
| 081 | H₃C | H₃C | H | CO₂CH₂CH₃ | CH | N | S | CH |
| 082 | H₃C | H₃C | H | CONH₂ | CH | N | S | CH |
| 083 | H₃C | H₃C | H | CONHCH₃ | CH | N | S | CH |
| 084 | H₃C | H₃C | H | CON(CH₃)₂ | CH | N | S | CH |
| 085 | H₃C | H₃C | CO₂H | H | CH | N | S | N |
| 086 | H₃C | H₃C | CO₂CH₃ | H | CH | N | S | N |
| 087 | H₃C | H₃C | CO₂CH₂CH₃ | H | CH | N | S | N |
| 088 | H₃C | H₃C | CONH₂ | H | CH | N | S | N |
| 089 | H₃C | H₃C | CONHCH₃ | H | CH | N | S | N |
| 090 | H₃C | H₃C | CON(CH₃)₂ | H | CH | N | S | N |
| 091 | H₃C | H₃C | H | CO₂H | CH | N | S | N |
| 092 | H₃C | H₃C | H | CO₂CH₃ | CH | N | S | N |
| 093 | H₃C | H₃C | H | CO₂CH₂CH₃ | CH | N | S | N |
| 094 | H₃C | H₃C | H | CONH₂ | CH | N | S | N |
| 095 | H₃C | H₃C | H | CONHCH₃ | CH | N | S | N |
| 096 | H₃C | H₃C | H | CON(CH₃)₂ | CH | N | S | N |
| 097 | H₃C | H₃C | CO₂H | H | CH | N | O | CH |
| 098 | H₃C | H₃C | CO₂CH₃ | H | CH | N | O | CH |
| 099 | H₃C | H₃C | CO₂CH₂CH₃ | H | CH | N | O | CH |
| 100 | H₃C | H₃C | CONH₂ | H | CH | N | O | CH |
| 101 | H₃C | H₃C | CONHCH₃ | H | CH | N | O | CH |
| 102 | H₃C | H₃C | CON(CH₃)₂ | H | CH | N | O | CH |
| 103 | H₃C | H₃C | H | CO₂H | CH | N | O | CH |
| 104 | H₃C | H₃C | H | CO₂CH₃ | CH | N | O | CH |
| 105 | H₃C | H₃C | H | CO₂CH₂CH₃ | CH | N | O | CH |
| 106 | H₃C | H₃C | H | CONH₂ | CH | N | O | CH |
| 107 | H₃C | H₃C | H | CONHCH₃ | CH | N | O | CH |
| 108 | H₃C | H₃C | H | CON(CH₃)₂ | CH | N | O | CH |
| 109 | H₃C | H₃C | CO₂H | H | N | N | S | CH |
| 110 | H₃C | H₃C | CO₂CH₃ | H | N | N | S | CH |
| 111 | H₃C | H₃C | CO₂CH₂CH₃ | H | N | N | S | CH |
| 112 | H₃C | H₃C | CONH₂ | H | N | N | S | CH |
| 113 | H₃C | H₃C | CONHCH₃ | H | N | N | S | CH |
| 114 | H₃C | H₃C | CON(CH₃)₂ | H | N | N | S | CH |
| 115 | H₃C | H₃C | H | CO₂H | N | N | S | CH |
| 116 | H₃C | H₃C | H | CO₂CH₃ | N | N | S | CH |
| 117 | H₃C | H₃C | H | CO₂CH₂CH₃ | N | N | S | CH |
| 118 | H₃C | H₃C | H | CONH₂ | N | N | S | CH |
| 119 | H₃C | H₃C | H | CONHCH₃ | N | N | S | CH |
| 120 | H₃C | H₃C | H | CON(CH₃)₂ | N | N | S | CH |
| 121 | H₃C | H₃C | CO₂H | H | N | N | S | N |
| 122 | H₃C | H₃C | CO₂CH₃ | H | N | N | S | N |
| 123 | H₃C | H₃C | CO₂CH₂CH₃ | H | N | N | S | N |
| 124 | H₃C | H₃C | CONH₂ | H | N | N | S | N |
| 125 | H₃C | H₃C | CONHCH₃ | H | N | N | S | N |
| 126 | H₃C | H₃C | CON(CH₃)₂ | H | N | N | S | N |
| 127 | H₃C | H₃C | H | CO₂H | N | N | S | N |
| 128 | H₃C | H₃C | H | CO₂CH₃ | N | N | S | N |
| 129 | H₃C | H₃C | H | CO₂CH₂CH₃ | N | N | S | N |
| 130 | H₃C | H₃C | H | CONH₂ | N | N | S | N |
| 131 | H₃C | H₃C | H | CONHCH₃ | N | N | S | N |
| 132 | H₃C | H₃C | H | CON(CH₃)₂ | N | N | S | N |
| 133 | H₃C | H₃C | CO₂H | H | N | N | O | CH |
| 134 | H₃C | H₃C | CO₂CH₃ | H | N | N | O | CH |
| 135 | H₃C | H₃C | CO₂CH₂CH₃ | H | N | N | O | CH |
| 136 | H₃C | H₃C | CONH₂ | H | N | N | O | CH |
| 137 | H₃C | H₃C | CONHCH₃ | H | N | N | O | CH |
| 138 | H₃C | H₃C | CON(CH₃)₂ | H | N | N | O | CH |
| 139 | H₃C | H₃C | H | CO₂H | N | N | O | CH |
| 140 | H₃C | H₃C | H | CO₂CH₃ | N | N | O | CH |
| 141 | H₃C | H₃C | H | CO₂CH₂CH₃ | N | N | O | CH |
| 142 | H₃C | H₃C | H | CONH₂ | N | N | O | CH |
| 143 | H₃C | H₃C | H | CONHCH₃ | N | N | O | CH |
| 144 | H₃C | H₃C | H | CON(CH₃)₂ | N | N | O | CH |
| 145 | F₂HC | H₃C | CO₂H | H | CH | N | S | CH |
| 146 | F₂HC | H₃C | CO₂CH₃ | H | CH | N | S | CH |
| 147 | F₂HC | H₃C | CO₂CH₂CH₃ | H | CH | N | S | CH |
| 148 | F₂HC | H₃C | CONH₂ | H | CH | N | S | CH |
| 149 | F₂HC | H₃C | CONHCH₃ | H | CH | N | S | CH |
| 150 | F₂HC | H₃C | CON(CH₃)₂ | H | CH | N | S | CH |
| 151 | F₂HC | H₃C | H | CO₂H | CH | N | S | CH |
| 152 | F₂HC | H₃C | H | CO₂CH₃ | CH | N | S | CH |
| 153 | F₂HC | H₃C | H | CO₂CH₂CH₃ | CH | N | S | CH |
| 154 | F₂HC | H₃C | H | CONH₂ | CH | N | S | CH |
| 155 | F₂HC | H₃C | H | CONHCH₃ | CH | N | S | CH |
| 156 | F₂HC | H₃C | H | CON(CH₃)₂ | CH | N | S | CH |
| 157 | F₂HC | H₃C | CO₂H | H | CH | N | S | N |
| 158 | F₂HC | H₃C | CO₂CH₃ | H | CH | N | S | N |
| 159 | F₂HC | H₃C | CO₂CH₂CH₃ | H | CH | N | S | N |
| 160 | F₂HC | H₃C | CONH₂ | H | CH | N | S | N |
| 161 | F₂HC | H₃C | CONHCH₃ | H | CH | N | S | N |
| 162 | F₂HC | H₃C | CON(CH₃)₂ | H | CH | N | S | N |
| 163 | F₂HC | H₃C | H | CO₂H | CH | N | S | N |
| 164 | F₂HC | H₃C | H | CO₂CH₃ | CH | N | S | N |
| 165 | F₂HC | H₃C | H | CO₂CH₂CH₃ | CH | N | S | N |
| 166 | F₂HC | H₃C | H | CONH₂ | CH | N | S | N |
| 167 | F₂HC | H₃C | H | CONHCH₃ | CH | N | S | N |
| 168 | F₂HC | H₃C | H | CON(CH₃)₂ | CH | N | S | N |
| 169 | F₂HC | H₃C | CO₂H | H | CH | N | O | CH |
| 170 | F₂HC | H₃C | CO₂CH₃ | H | CH | N | O | CH |
| 171 | F₂HC | H₃C | CO₂CH₂CH₃ | H | CH | N | O | CH |
| 172 | F₂HC | H₃C | CONH₂ | H | CH | N | O | CH |
| 173 | F₂HC | H₃C | CONHCH₃ | H | CH | N | O | CH |
| 174 | F₂HC | H₃C | CON(CH₃)₂ | H | CH | N | O | CH |
| 175 | F₂HC | H₃C | H | CO₂H | CH | N | O | CH |
| 176 | F₂HC | H₃C | H | CO₂CH₃ | CH | N | O | CH |
| 177 | F₂HC | H₃C | H | CO₂CH₂CH₃ | CH | N | O | CH |
| 178 | F₂HC | H₃C | H | CONH₂ | CH | N | O | CH |
| 179 | F₂HC | H₃C | H | CONHCH₃ | CH | N | O | CH |
| 180 | F₂HC | H₃C | H | CON(CH₃)₂ | CH | N | O | CH |
| 181 | F₂HC | H₃C | CO₂H | H | N | N | S | CH |
| 182 | F₂HC | H₃C | CO₂CH₃ | H | N | N | S | CH |
| 183 | F₂HC | H₃C | CO₂CH₂CH₃ | H | N | N | S | CH |
| 184 | F₂HC | H₃C | CONH₂ | H | N | N | S | CH |
| 185 | F₂HC | H₃C | CONHCH₃ | H | N | N | S | CH |
| 186 | F₂HC | H₃C | CON(CH₃)₂ | H | N | N | S | CH |
| 187 | F₂HC | H₃C | H | CO₂H | N | N | S | CH |
| 188 | F₂HC | H₃C | H | CO₂CH₃ | N | N | S | CH |
| 189 | F₂HC | H₃C | H | CO₂CH₂CH₃ | N | N | S | CH |
| 190 | F₂HC | H₃C | H | CONH₂ | N | N | S | CH |
| 191 | F₂HC | H₃C | H | CONHCH₃ | N | N | S | CH |
| 192 | F₂HC | H₃C | H | CON(CH₃)₂ | N | N | S | CH |
| 193 | F₂HC | H₃C | CO₂H | H | N | N | S | N |
| 194 | F₂HC | H₃C | CO₂CH₃ | H | N | N | S | N |
| 195 | F₂HC | H₃C | CO₂CH₂CH₃ | H | N | N | S | N |
| 196 | F₂HC | H₃C | CONH₂ | H | N | N | S | N |
| 197 | F₂HC | H₃C | CONHCH₃ | H | N | N | S | N |
| 198 | F₂HC | H₃C | CON(CH₃)₂ | H | N | N | S | N |
| 199 | F₂HC | H₃C | H | CO₂H | N | N | S | N |
| 200 | F₂HC | H₃C | H | CO₂CH₃ | N | N | S | N |
| 201 | F₂HC | H₃C | H | CO₂CH₂CH₃ | N | N | S | N |
| 202 | F₂HC | H₃C | H | CONH₂ | N | N | S | N |
| 203 | F₂HC | H₃C | H | CONHCH₃ | N | N | S | N |
| 204 | F₂HC | H₃C | H | CON(CH₃)₂ | N | N | S | N |
| 205 | F₂HC | H₃C | CO₂H | H | N | N | O | CH |
| 206 | F₂HC | H₃C | CO₂CH₃ | H | N | N | O | CH |
| 207 | F₂HC | H₃C | CO₂CH₂CH₃ | H | N | N | O | CH |
| 208 | F₂HC | H₃C | CONH₂ | H | N | N | O | CH |
| 209 | F₂HC | H₃C | CONHCH₃ | H | N | N | O | CH |
| 210 | F₂HC | H₃C | CON(CH₃)₂ | H | N | N | O | CH |
| 211 | F₂HC | H₃C | H | CO₂H | N | N | O | CH |
| 212 | F₂HC | H₃C | H | CO₂CH₃ | N | N | O | CH |
| 213 | F₂HC | H₃C | H | CO₂CH₂CH₃ | N | N | O | CH |
| 214 | F₂HC | H₃C | H | CONH₂ | N | N | O | CH |
| 215 | F₂HC | H₃C | H | CONHCH₃ | N | N | O | CH |
| 216 | F₂HC | H₃C | H | CON(CH₃)₂ | N | N | O | CH |
| 217 | F₂HC | F₂HC | CO₂H | H | CH | N | S | CH |
| 218 | F₂HC | F₂HC | CO₂CH₃ | H | CH | N | S | CH |
| 219 | F₂HC | F₂HC | CO₂CH₂CH₃ | H | CH | N | S | CH |
| 220 | F₂HC | F₂HC | CONH₂ | H | CH | N | S | CH |
| 221 | F₂HC | F₂HC | CONHCH₃ | H | CH | N | S | CH |
| 222 | F₂HC | F₂HC | CON(CH₃)₂ | H | CH | N | S | CH |
| 223 | F₂HC | F₂HC | H | CO₂H | CH | N | S | CH |
| 224 | F₂HC | F₂HC | H | CO₂CH₃ | CH | N | S | CH |
| 225 | F₂HC | F₂HC | H | CO₂CH₂CH₃ | CH | N | S | CH |
| 226 | F₂HC | F₂HC | H | CONH₂ | CH | N | S | CH |
| 227 | F₂HC | F₂HC | H | CONHCH₃ | CH | N | S | CH |
| 228 | F₂HC | F₂HC | H | CON(CH₃)₂ | CH | N | S | CH |
| 229 | F₂HC | F₂HC | CO₂H | H | CH | N | S | N |
| 230 | F₂HC | F₂HC | CO₂CH₃ | H | CH | N | S | N |
| 231 | F₂HC | F₂HC | CO₂CH₂CH₃ | H | CH | N | S | N |
| 232 | F₂HC | F₂HC | CONH₂ | H | CH | N | S | N |
| 233 | F₂HC | F₂HC | CONHCH₃ | H | CH | N | S | N |
| 234 | F₂HC | F₂HC | CON(CH₃)₂ | H | CH | N | S | N |
| 235 | F₂HC | F₂HC | H | CO₂H | CH | N | S | N |
| 236 | F₂HC | F₂HC | H | CO₂CH₃ | CH | N | S | N |
| 237 | F₂HC | F₂HC | H | CO₂CH₂CH₃ | CH | N | S | N |
| 238 | F₂HC | F₂HC | H | CONH₂ | CH | N | S | N |
| 239 | F₂HC | F₂HC | H | CONHCH₃ | CH | N | S | N |
| 240 | F₂HC | F₂HC | H | CON(CH₃)₂ | CH | N | S | N |
| 241 | F₂HC | F₂HC | CO₂H | H | CH | N | O | CH |
| 242 | F₂HC | F₂HC | CO₂CH₃ | H | CH | N | O | CH |
| 243 | F₂HC | F₂HC | CO₂CH₂CH₃ | H | CH | N | O | CH |
| 244 | F₂HC | F₂HC | CONH₂ | H | CH | N | O | CH |
| 245 | F₂HC | F₂HC | CONHCH₃ | H | CH | N | O | CH |
| 246 | F₂HC | F₂HC | CON(CH₃)₂ | H | CH | N | O | CH |
| 247 | F₂HC | F₂HC | H | CO₂H | CH | N | O | CH |
| 248 | F₂HC | F₂HC | H | CO₂CH₃ | CH | N | O | CH |
| 249 | F₂HC | F₂HC | H | CO₂CH₂CH₃ | CH | N | O | CH |
| 250 | F₂HC | F₂HC | H | CONH₂ | CH | N | O | CH |
| 251 | F₂HC | F₂HC | H | CONHCH₃ | CH | N | O | CH |
| 252 | F₂HC | F₂HC | H | CON(CH₃)₂ | CH | N | O | CH |
| 253 | F₂HC | F₂HC | CO₂H | H | N | N | S | CH |
| 254 | F₂HC | F₂HC | CO₂CH₃ | H | N | N | S | CH |
| 255 | F₂HC | F₂HC | CO₂CH₂CH₃ | H | N | N | S | CH |
| 256 | F₂HC | F₂HC | CONH₂ | H | N | N | S | CH |
| 257 | F₂HC | F₂HC | CONHCH₃ | H | N | N | S | CH |
| 258 | F₂HC | F₂HC | CON(CH₃)₂ | H | N | N | S | CH |
| 259 | F₂HC | F₂HC | H | CO₂H | N | N | S | CH |
| 260 | F₂HC | F₂HC | H | CO₂CH₃ | N | N | S | CH |
| 261 | F₂HC | F₂HC | H | CO₂CH₂CH₃ | N | N | S | CH |
| 262 | F₂HC | F₂HC | H | CONH₂ | N | N | S | CH |
| 263 | F₂HC | F₂HC | H | CONHCH₃ | N | N | S | CH |
| 264 | F₂HC | F₂HC | H | CON(CH₃)₂ | N | N | S | CH |
| 265 | F₂HC | F₂HC | CO₂H | H | N | N | S | N |
| 266 | F₂HC | F₂HC | CO₂CH₃ | H | N | N | S | N |
| 267 | F₂HC | F₂HC | CO₂CH₂CH₃ | H | N | N | S | N |
| 268 | F₂HC | F₂HC | CONH₂ | H | N | N | S | N |
| 269 | F₂HC | F₂HC | CONHCH₃ | H | N | N | S | N |
| 270 | F₂HC | F₂HC | CON(CH₃)₂ | H | N | N | S | N |
| 271 | F₂HC | F₂HC | H | CO₂H | N | N | S | N |
| 272 | F₂HC | F₂HC | H | CO₂CH₃ | N | N | S | N |
| 273 | F₂HC | F₂HC | H | CO₂CH₂CH₃ | N | N | S | N |
| 274 | F₂HC | F₂HC | H | CONH₂ | N | N | S | N |
| 275 | F₂HC | F₂HC | H | CONHCH₃ | N | N | S | N |
| 276 | F₂HC | F₂HC | H | CON(CH₃)₂ | N | N | S | N |
| 277 | F₂HC | F₂HC | CO₂H | H | N | N | O | CH |
| 278 | F₂HC | F₂HC | CO₂CH₃ | H | N | N | O | CH |
| 279 | F₂HC | F₂HC | CO₂CH₂CH₃ | H | N | N | O | CH |
| 280 | F₂HC | F₂HC | CONH₂ | H | N | N | O | CH |
| 281 | F₂HC | F₂HC | CONHCH₃ | H | N | N | O | CH |
| 282 | F₂HC | F₂HC | CON(CH₃)₂ | H | N | N | O | CH |
| 283 | F₂HC | F₂HC | H | CO₂H | N | N | O | CH |
| 284 | F₂HC | F₂HC | H | CO₂CH₃ | N | N | O | CH |
| 285 | F₂HC | F₂HC | H | CO₂CH₂CH₃ | N | N | O | CH |
| 286 | F₂HC | F₂HC | H | CONH₂ | N | N | O | CH |
| 287 | F₂HC | F₂HC | H | CONHCH₃ | N | N | O | CH |
| 288 | F₂HC | F₂HC | H | CON(CH₃)₂ | N | N | O | CH |
| 289 | F₃C | F₃C | CO₂H | H | CH | N | S | CH |
| 290 | F₃C | F₃C | CO₂CH₃ | H | CH | N | S | CH |
| 291 | F₃C | F₃C | CO₂CH₂CH₃ | H | CH | N | S | CH |
| 292 | F₃C | F₃C | CONH₂ | H | CH | N | S | CH |
| 293 | F₃C | F₃C | CONHCH₃ | H | CH | N | S | CH |
| 294 | F₃C | F₃C | CON(CH₃)₂ | H | CH | N | S | CH |
| 295 | F₃C | F₃C | H | CO₂H | CH | N | S | CH |
| 296 | F₃C | F₃C | H | CO₂CH₃ | CH | N | S | CH |
| 297 | F₃C | F₃C | H | CO₂CH₂CH₃ | CH | N | S | CH |
| 298 | F₃C | F₃C | H | CONH₂ | CH | N | S | CH |
| 299 | F₃C | F₃C | H | CONHCH₃ | CH | N | S | CH |
| 300 | F₃C | F₃C | H | CON(CH₃)₂ | CH | N | S | CH |
| 301 | F₃C | F₃C | CO₂H | H | CH | N | S | N |
| 302 | F₃C | F₃C | CO₂CH₃ | H | CH | N | S | N |
| 303 | F₃C | F₃C | CO₂CH₂CH₃ | H | CH | N | S | N |
| 304 | F₃C | F₃C | CONH₂ | H | CH | N | S | N |
| 305 | F₃C | F₃C | CONHCH₃ | H | CH | N | S | N |
| 306 | F₃C | F₃C | CON(CH₃)₂ | H | CH | N | S | N |
| 307 | F₃C | F₃C | H | CO₂H | CH | N | S | N |
| 308 | F₃C | F₃C | H | CO₂CH₃ | CH | N | S | N |
| 309 | F₃C | F₃C | H | CO₂CH₂CH₃ | CH | N | S | N |
| 310 | F₃C | F₃C | H | CONH₂ | CH | N | S | N |
| 311 | F₃C | F₃C | H | CONHCH₃ | CH | N | S | N |
| 312 | F₃C | F₃C | H | CON(CH₃)₂ | CH | N | S | N |
| 313 | F₃C | F₃C | CO₂H | H | CH | N | O | CH |
| 314 | F₃C | F₃C | CO₂CH₃ | H | CH | N | O | CH |
| 315 | F₃C | F₃C | CO₂CH₂CH₃ | H | CH | N | O | CH |
| 316 | F₃C | F₃C | CONH₂ | H | CH | N | O | CH |
| 317 | F₃C | F₃C | CONHCH₃ | H | CH | N | O | CH |
| 318 | F₃C | F₃C | CON(CH₃)₂ | H | CH | N | O | CH |
| 319 | F₃C | F₃C | H | CO₂H | CH | N | O | CH |
| 320 | F₃C | F₃C | H | CO₂CH₃ | CH | N | O | CH |
| 321 | F₃C | F₃C | H | CO₂CH₂CH₃ | CH | N | O | CH |
| 322 | F₃C | F₃C | H | CONH₂ | CH | N | O | CH |
| 323 | F₃C | F₃C | H | CONHCH₃ | CH | N | O | CH |
| 324 | F₃C | F₃C | H | CON(CH₃)₂ | CH | N | O | CH |
| 325 | F₃C | F₃C | CO₂H | H | N | N | S | CH |
| 326 | F₃C | F₃C | CO₂CH₃ | H | N | N | S | CH |
| 327 | F₃C | F₃C | CO₂CH₂CH₃ | H | N | N | S | CH |
| 328 | F₃C | F₃C | CONH₂ | H | N | N | S | CH |
| 329 | F₃C | F₃C | CONHCH₃ | H | N | N | S | CH |
| 330 | F₃C | F₃C | CON(CH₃)₂ | H | N | N | S | CH |
| 331 | F₃C | F₃C | H | CO₂H | N | N | S | CH |
| 332 | F₃C | F₃C | H | CO₂CH₃ | N | N | S | CH |
| 333 | F₃C | F₃C | H | CO₂CH₂CH₃ | N | N | S | CH |
| 334 | F₃C | F₃C | H | CONH₂ | N | N | S | CH |
| 335 | F₃C | F₃C | H | CONHCH₃ | N | N | S | CH |
| 336 | F₃C | F₃C | H | CON(CH₃)₂ | N | N | S | CH |
| 337 | F₃C | F₃C | CO₂H | H | N | N | S | N |
| 338 | F₃C | F₃C | CO₂CH₃ | H | N | N | S | N |
| 339 | F₃C | F₃C | CO₂CH₂CH₃ | H | N | N | S | N |
| 340 | F₃C | F₃C | CONH₂ | H | N | N | S | N |
| 341 | F₃C | F₃C | CONHCH₃ | H | N | N | S | N |
| 342 | F₃C | F₃C | CON(CH₃)₂ | H | N | N | S | N |
| 343 | F₃C | F₃C | H | CO₂H | N | N | S | N |
| 344 | F₃C | F₃C | H | CO₂CH₃ | N | N | S | N |
| 345 | F₃C | F₃C | H | CO₂CH₂CH₃ | N | N | S | N |
| 346 | F₃C | F₃C | H | CONH₂ | N | N | S | N |
| 347 | F₃C | F₃C | H | CONHCH₃ | N | N | S | N |
| 348 | F₃C | F₃C | H | CON(CH₃)₂ | N | N | S | N |
| 349 | F₃C | F₃C | CO₂H | H | N | N | O | CH |
| 350 | F₃C | F₃C | CO₂CH₃ | H | N | N | O | CH |
| 351 | F₃C | F₃C | CO₂CH₂CH₃ | H | N | N | O | CH |
| 352 | F₃C | F₃C | CONH₂ | H | N | N | O | CH |
| 353 | F₃C | F₃C | CONHCH₃ | H | N | N | O | CH |
| 354 | F₃C | F₃C | CON(CH₃)₂ | H | N | N | O | CH |
| 355 | F₃C | F₃C | H | CO₂H | N | N | O | CH |
| 356 | F₃C | F₃C | H | CO₂CH₃ | N | N | O | CH |
| 357 | F₃C | F₃C | H | CO₂CH₂CH₃ | N | N | O | CH |
| 358 | F₃C | F₃C | H | CONH₂ | N | N | O | CH |
| 359 | F₃C | F₃C | H | CONHCH₃ | N | N | O | CH |
| 360 | F₃C | F₃C | H | CON(CH₃)₂ | N | N | O | CH |

where
a) 360 compounds of formula (I.a):

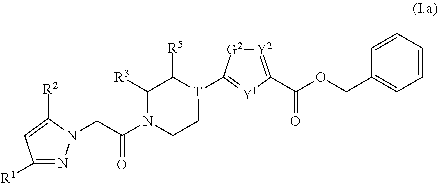

wherein R¹, R², R³, R⁵, G², T, Y¹ and Y² are as defined in Table 1.
b) 360 compounds of formula (I.b):

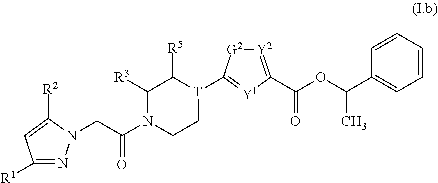

wherein R¹, R², R³, R⁵, G², T, Y¹ and Y² are as defined in Table 1.
c) 360 compounds of formula (I.c):

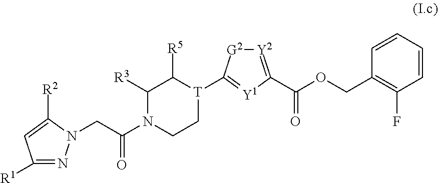

wherein R¹, R², R³, R⁵, G², T, Y¹ and Y² are as defined in Table 1.
d) 360 compounds of formula (I.d):

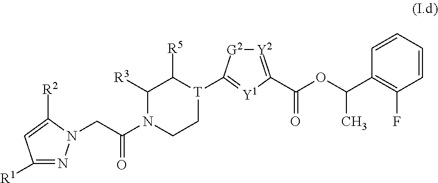

Wherein R¹, R², R³, R⁵, G², T, Y¹ and Y² are as defined in Table 1.
e) 360 compounds of formula (I.e):

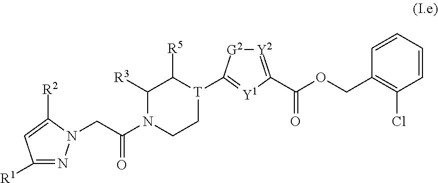

wherein R¹, R², R³, R⁵, G², T, Y¹ and Y² are as defined in Table 1.
f) 360 compounds of formula (I.f):

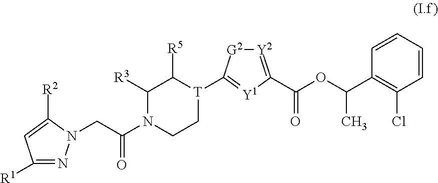

wherein R¹, R², R³, R⁵, G², T, Y¹ and Y² are as defined in Table 1.
g) 360 compounds of formula (I.g):

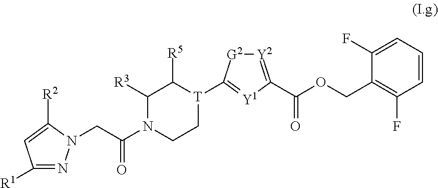

wherein R¹, R², R³, R⁵, G², T, Y¹ and Y² are as defined in Table 1.
h) 360 compounds of formula (I.h):

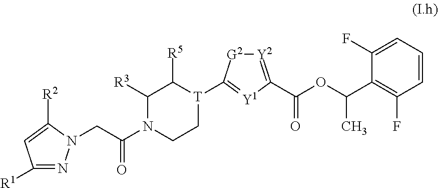

wherein R¹, R², R³, R⁵, G², T, Y¹ and Y² are as defined in Table 1.
i) 360 compounds of formula (I.i):

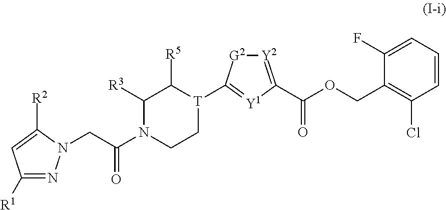

wherein R¹, R², R³, R⁵, G², T, Y¹ and Y² are as defined in Table 1.
j) 360 compounds of formula (I.j):

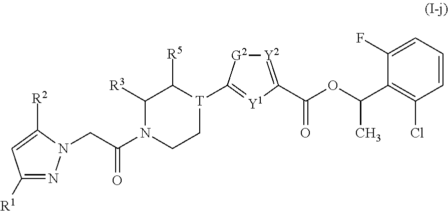

wherein R¹, R², R³, R⁵, G², T, Y¹ and Y² are as defined in Table 1.
k) 360 compounds of formula (I.k):

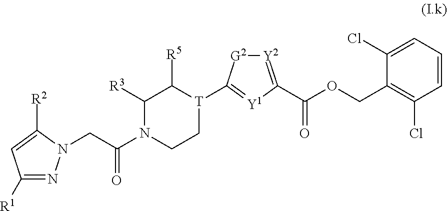

wherein R¹, R², R³, R⁵, G², T, Y¹ and Y² are as defined in Table 1.
m) 360 compounds of formula (I.m):

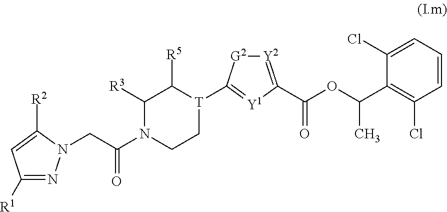

wherein R¹, R², R³, R⁵, G², T, Y¹ and Y² are as defined in Table 1.
n) 360 compounds of formula (I.n):

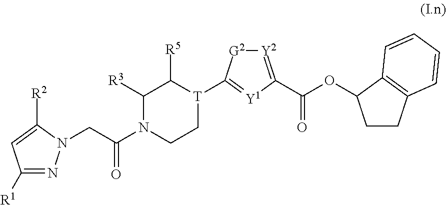

wherein R¹, R², R³, R⁵, G², T, Y¹ and Y² are as defined in Table 1.
o) 360 compounds of formula (I.o):

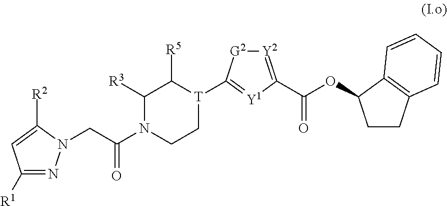

wherein R¹, R², R³, R⁵, G², T, Y¹ and Y² are as defined in Table 1.
p) 360 compounds of formula (I.p):

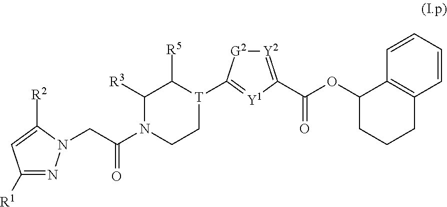

wherein R¹, R², R³, R⁵, G², T, Y¹ and Y² are as defined in Table 1.
q) 360 compounds of formula (I.q):

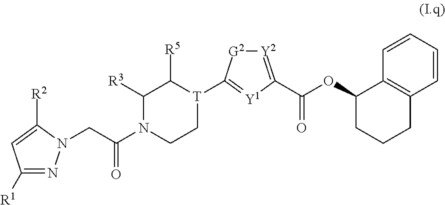

wherein R¹, R², R³, R⁵, G², T, Y¹ and Y² are as defined in Table 1.
r) 360 compounds of formula (I.r):

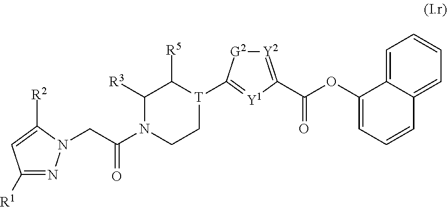

wherein R¹, R², R³, R⁵, G², T, Y¹ and Y² are as defined in Table 1.
s) 360 compounds of formula (I.s):

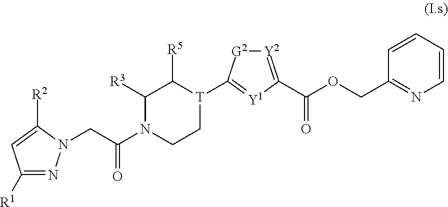

wherein R¹, R², R³, R⁵, G², T, Y¹ and Y² are as defined in Table 1.
t) 360 compounds of formula (I.t):

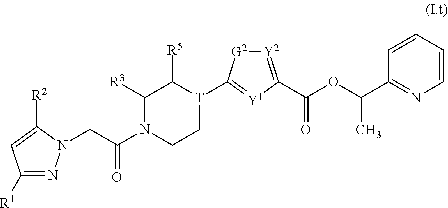

wherein R¹, R², R³, R⁵, G², T, Y¹ and Y² are as defined in Table 1.
u) 360 compounds of formula (I.u):

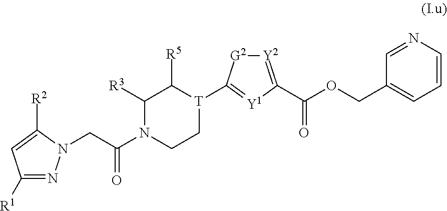

wherein R¹, R², R³, R⁵, G², T, Y¹ and Y² are as defined in Table 1.
v) 360 compounds of formula (I.v):

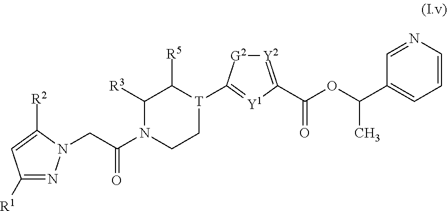

wherein R¹, R², R³, R⁵, G², T, Y¹ and Y² are as defined in Table 1.
w) 360 compounds of formula (I.w):

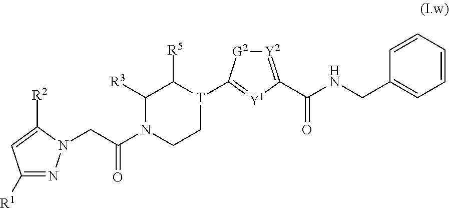

wherein R¹, R², R³, R⁵, G², T, Y¹ and Y² are as defined in Table 1.
x) 360 compounds of formula (I.x):

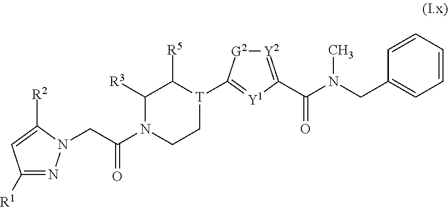

wherein R¹, R², R³, R⁵, G², T, Y¹ and Y² are as defined in Table 1.
y) 360 compounds of formula (I.y):

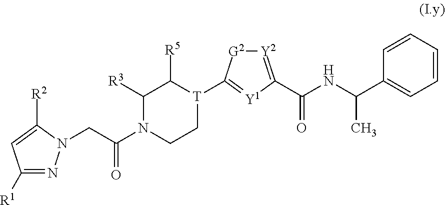

wherein R¹, R², R³, R⁵, G², T, Y¹ and Y² are as defined in Table 1.
z) 360 compounds of formula (I.z):

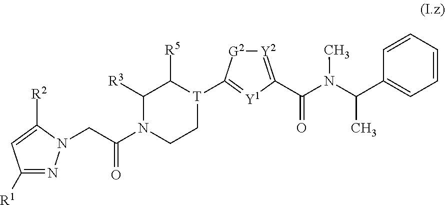

wherein R¹, R², R³, R⁵, G², T, Y¹ and Y² are as defined in Table 1.
aa) 360 compounds of formula (I.aa):

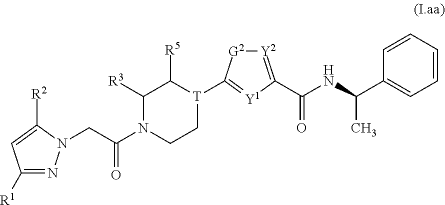

wherein R¹, R², R³, R⁵, G², T, Y¹ and Y² are as defined in Table 1.
ab) 360 compounds of formula (I.ab):

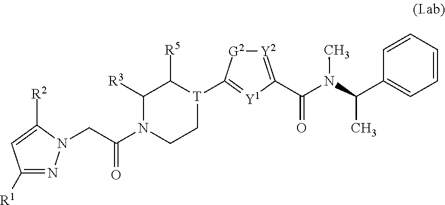

wherein R¹, R², R³, R⁵, G², T, Y¹ and Y² are as defined in Table 1.
ac) 360 compounds of formula (I.ac):

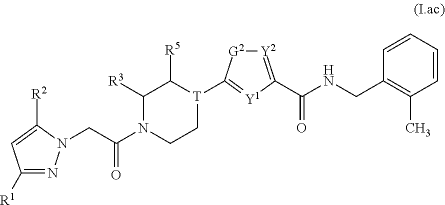

wherein R¹, R², R³, R⁵, G², T, Y¹ and Y² are as defined in Table 1.
ad) 360 compounds of formula (I.ad):

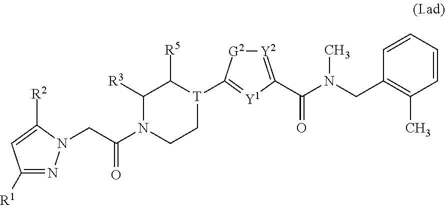

wherein R¹, R², R³, R⁵, G², T, Y¹ and Y² are as defined in Table 1.
ae) 360 compounds of formula (I.ae):

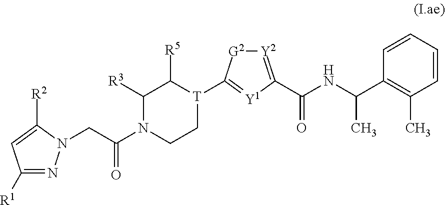

wherein R¹, R², R³, R⁵, G², T, Y¹ and Y² are as defined in Table 1.
af) 360 compounds of formula (I.af):

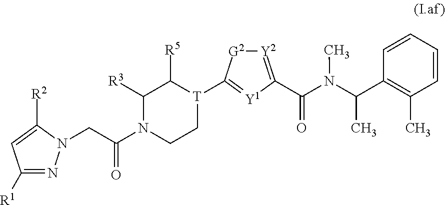

wherein R¹, R², R³, R⁵, G², T, Y¹ and Y² are as defined in Table 1.
ag) 360 compounds of formula (I.ag):

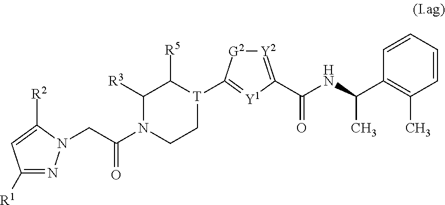

wherein R¹, R², R³, R⁵, G², T, Y¹ and Y² are as defined in Table 1.
ah) 360 compounds of formula (I.ah):

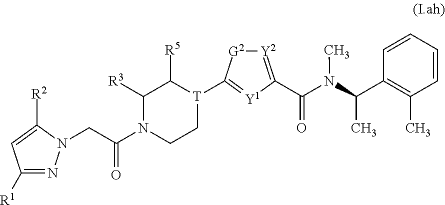

wherein R¹, R², R³, R⁵, G², T, Y¹ and Y² are as defined in Table 1.
ai) 360 compounds of formula (I.ai):

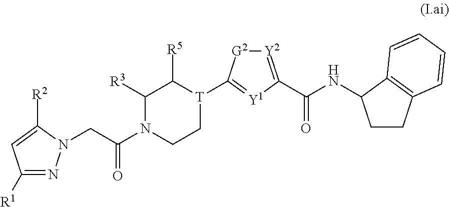

wherein R¹, R², R³, R⁵, G², T, Y¹ and Y² are as defined in Table 1.
aj) 360 compounds of formula (I.aj):

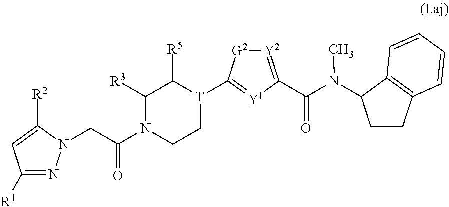

wherein R¹, R², R³, R⁵, G², T, Y¹ and Y² are as defined in Table 1.
ak) 360 compounds of formula (I.ak):

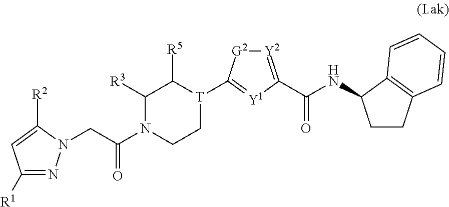

wherein R¹, R², R³, R⁵, G², T, Y¹ and Y² are as defined in Table 1.
am) 360 compounds of formula (I.am):

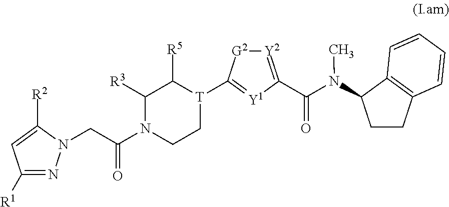

wherein R¹, R², R³, R⁵, G², T, Y¹ and Y² are as defined in Table 1.
an) 360 compounds of formula (I.an):

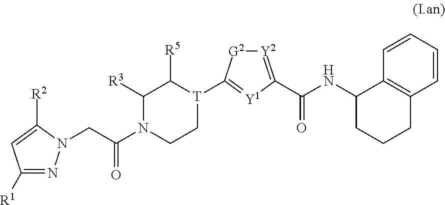

wherein R¹, R², R³, R⁵, G², T, Y¹ and Y² are as defined in Table 1.
ao) 360 compounds of formula (I.ao):

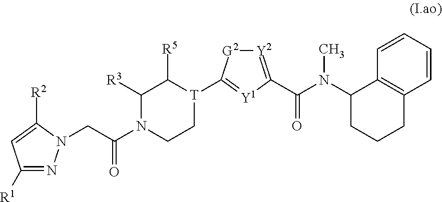

wherein R¹, R², R³, R⁵, G², T, Y¹ and Y² are as defined in Table 1.
ap) 360 compounds of formula (I.ap):

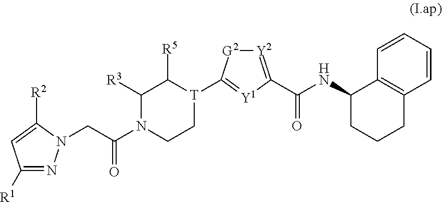

wherein R¹, R², R³, R⁵, G², T, Y¹ and Y² are as defined in Table 1.
aq) 360 compounds of formula (I.aq):

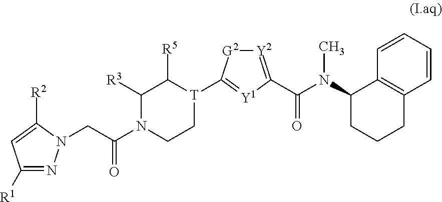

wherein R¹, R², R³, R⁵, G², T, Y¹ and Y² are as defined in Table 1.
ar) 360 compounds of formula (I.ar):

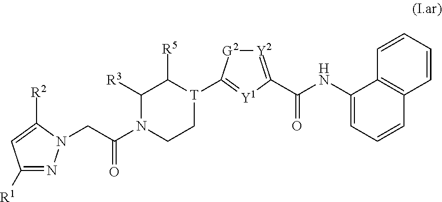

wherein R¹, R², R³, R⁵, G², T, Y¹ and Y² are as defined in Table 1.
as) 360 compounds of formula (I.as):

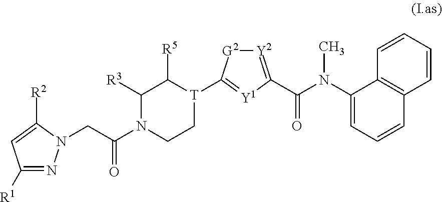

wherein R¹, R², R³, R⁵, G², T, Y¹ and Y² are as defined in Table 1.
at) 360 compounds of formula (I.at):

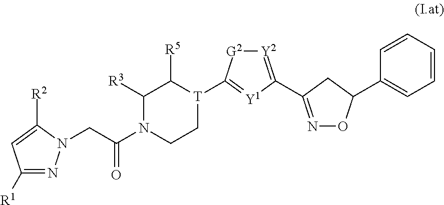

wherein R¹, R², R³, R⁵, G², T, Y¹ and Y² are as defined in Table 1.
au) 360 compounds of formula (I.au):

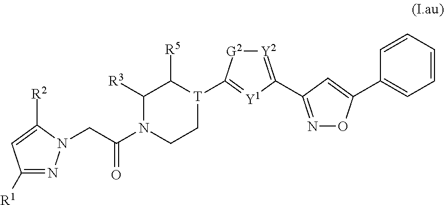

wherein R¹, R², R³, R⁵, G², T, Y¹ and Y² are as defined in Table 1.
av) 360 compounds of formula (I.av):

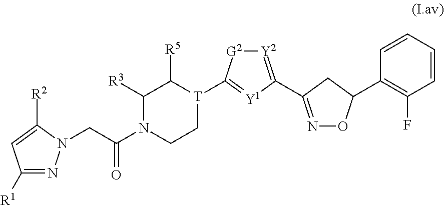

wherein R¹, R², R³, R⁵, G², T, Y¹ and Y² are as defined in Table 1.
aw) 360 compounds of formula (I.aw):

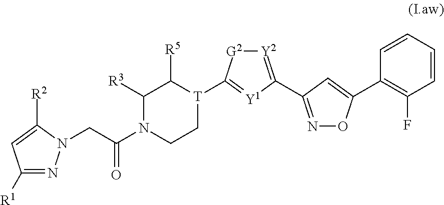

wherein R¹, R², R³, R⁵, G², T, Y¹ and Y² are as defined in Table 1.
ax) 360 compounds of formula (I.ax):

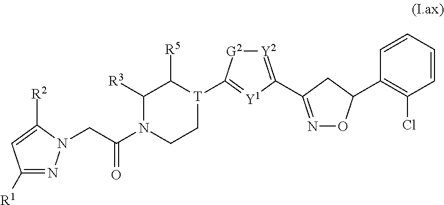

wherein R¹, R², R³, R⁵, G², T, Y¹ and Y² are as defined in Table 1.
ay) 360 compounds of formula (I.ay):

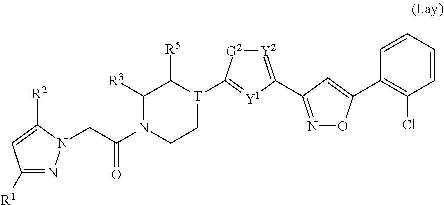

Wherein R¹, R², R³, R⁵, G², T, Y¹ and Y² are as defined in Table 1.
az) 360 compounds of formula (I.az):

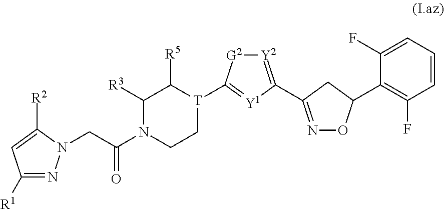

wherein R¹, R², R³, R⁵, G², T, Y¹ and Y² are as defined in Table 1.
ba) 360 compounds of formula (I.ba):

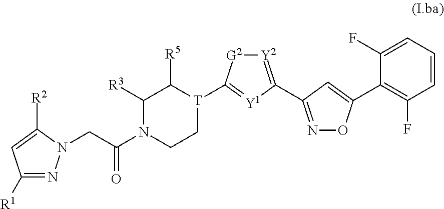

wherein R¹, R², R³, R⁵, G², T, Y¹ and Y² are as defined in Table 1.
bb) 360 compounds of formula (I.bb):

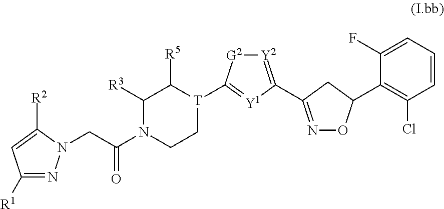

wherein R¹, R², R³, R⁵, G², T, Y¹ and Y² are as defined in Table 1.
bc) 360 compounds of formula (I.bc):

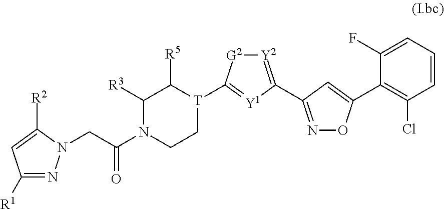

wherein R¹, R², R³, R⁵, G², T, Y¹ and Y² are as defined in Table 1.
bd) 360 compounds of formula (I.bd):

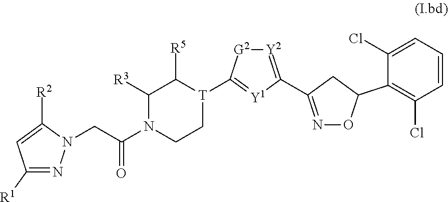

wherein R¹, R², R³, R⁵, G², T, Y¹ and Y² are as defined in Table 1.
be) 360 compounds of formula (I.be):

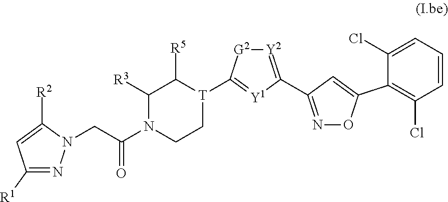

wherein R¹, R², R³, R⁵, G², T, Y¹ and Y² are as defined in Table 1.
bf) 360 compounds of formula (I.bf):

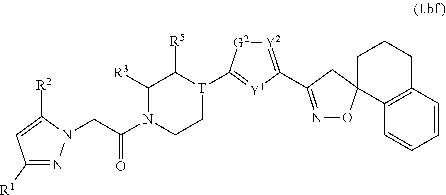

wherein R¹, R², R³, R⁵, G², T, Y¹ and Y² are as defined in Table 1.

Throughout this description, temperatures are given in degrees Celsius and “m.p.” means melting point. LC/MS means Liquid Chromatography Mass Spectroscopy and the description of the apparatus and the method is: (ACQUITY UPLC from Waters, Phenomenex Gemini C18, 3 μm particle size, 110 Angström, 30×3 mm column, 1.7 mL/min., 60° C., H₂O+0.05% HCOOH (95%)/CH₃CN/MeOH 4:1+0.04% HCOOH (5%)−2 min.−CH₃CN/MeOH 4:1+0.04% HCOOH (5%)−0.8 min., ACQUITY SQD Mass Spectrometer from Waters, ionization method: electrospray (ESI), Polarity: positive ions, Capillary (kV) 3.00, Cone (V) 20.00, Extractor (V) 3.00, Source Temperature (° C.) 150, Desolvation Temperature (° C.) 400, Cone Gas Flow (L/Hr) 60, Desolvation Gas Flow (L/Hr) 700)).

TABLE 2
Melting point and LC/MS data for compounds of Table 1

| Compound | Melting point | |
|---|---|---|
| No. | (° C.) | LC/MS |
| I.az.037 | 178-185 | |
| I.az.038 | | Rt = 1.70 min; MS: m/z = 599.4 (M + 1) |
| I.az.254 | | Rt = 1.66 min; MS: m/z = 617.3 (M + 1) |
| I.az.043 | | Rt = 1.49 min; MS: m/z = 585.4 (M + 1) |
| I.az.044 | | Rt = 1.69 min; MS: m/z = 599.4 (M + 1) |
| I.az.259 | | Rt = 1.46 min; MS: m/z = 603.4 (M + 1) |
| I.az.260 | | Rt = 1.65 min; MS: m/z = 617.4 (M + 1) |

The compounds according to the present invention can be prepared according to the above-mentioned reaction schemes, in which, unless otherwise stated, the definition of each variable is as defined above for a compound of formula (I).

### Biological Examples

Phytophthora Infestans/Tomato/Leaf Disc Preventative (Tomato Late Blight)

Tomato leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 16° C. and 75% rh under a light regime of 24 h darkness followed by 12 h light/12 h darkness in a climate cabinet and the activity of a compound is assessed as percent disease control compared to untreated when an appropriate level of disease damage appears in untreated check leaf disks (5-7 days after application).

Compounds I.az.038, I.az.044, I.az.254, I.az.259 and I.az.260 at 200 ppm give at least 80% disease control in this test when compared to untreated control leaf disks under the same conditions, which show extensive disease development.

Phytophthora infestans/Potato/Preventative (Potato Late Blight)

2-week old potato plants cv. Bintje are sprayed in a spray chamber with the formulated test compound diluted in water. The test plants are inoculated by spraying them with a sporangia suspension 2 days after application. The inoculated test plants are incubated at 18° C. with 14 h light/day and 100% rh in a growth chamber and the percentage leaf area covered by disease is assessed when an appropriate level of disease appears on untreated check plants (5-7 days after application).

Phytophthora infestans/Potato/Curative (Potato Late Blight)

2-week old potato plants cv. Bintje are inoculated by spraying them with a sporangia suspension one day before application. The inoculated plants are sprayed in a spray chamber with the formulated test compound diluted in water. The inoculated test plants are incubated at 18° C. with 14 h light/day and 100% rh in a growth chamber and the percentage leaf area covered by disease is assessed when an appropriate level of disease appears on untreated check plants (3-4 days after application).

Phytophthora infestans I Potato/Long Lasting (Potato Late Blight)

2-week old potato plants cv. Bintje are sprayed in a spray chamber with the formulated test compound diluted in water. The test plants are inoculated by spraying them with a sporangia suspension 6 days after application. The inoculated test plants are incubated at 18° C. with 14 h light/day and 100% rh in a growth chamber and the percentage leaf area covered by disease is assessed when an appropriate level of disease appears on untreated check plants (9-11 days after application).

Plasmopara viticola/Grape/Leaf Disc Preventative (Grape Downy Mildew)

Grape vine leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 19° C. and 80% rh under a light regime of 12 h light/12 h darkness in a climate cabinet and the activity of a compound is assessed as percent disease control compared to untreated when an appropriate level of disease damage appears in untreated check leaf disks (6-8 days after application).

Compounds I.az.038, I.az.254 and I.az.260 at 200 ppm give at least 80% disease control in this test when compared to untreated control leaf disks under the same conditions, which show extensive disease development.

Plasmopara viticola/Grape/Preventative (Rape Downy Mildew)

5-week old grape seedlings cv. Gutedel are sprayed in a spray chamber with the formulated test compound diluted in water. The test plants plants are inoculated by spraying a sporangia suspension on their lower leaf surface one day after application. The inoculated test plants are incubated at 22° C. and 100% rh in a greenhouse and the percentage leaf area covered by disease is assessed when an appropriate level of disease appears on untreated check plants (6-8 days after application).

Plasmopara viticola/Grape/Curative (Grape Downy Mildew)

5-week-old grape seedlings cv. Gutedel are inoculated by spraying a sporangia suspension on their lower leaf surface one day before application. The inoculated grape plants are sprayed in a spray chamber with the formulated test compound diluted in water. The inoculated test plants are incubated at 22° C. and 100% rh in a greenhouse and the percentage leaf area covered by disease is assessed when an appropriate level of disease appears on untreated check plants (4-6 days after application).

Plasmopara viticola/Grape/Long Lasting (Grape Downy Mildew)

5-week old grape seedlings cv. Gutedel are sprayed in a spray chamber with the formulated test compound diluted in water. The test plants are inoculated by spraying a sporangia suspension on their lower leaf surface 6 days after application. The inoculated test plants are incubated at 22° C. and 100% rh in a greenhouse and the percentage leaf area covered by disease is assessed when an appropriate level of disease appears on untreated check plants (11-13 days after application).

Pythium ultimum/Liquid Culture (Seedling Damping Off)

Mycelia fragments and oospores of a newly grown liquid culture of the fungus are directly mixed into nutrient broth (PDB potato dextrose broth). After placing a (DMSO) solution of test compound into a microtiter plate (96-well format), the nutrient broth containing the fungal mycelia/spore mixture is added. The test plates are incubated at 24° C. and the inhibition of growth is determined photometrically 2-3 days after application.

Compounds I.az.038, I.az.043, I.az.044, I.az.254, Iaz.259 and I.az.260 at 200 ppm give at least 80% disease control in this test when compared to untreated control agar droplets under the same conditions, which show extensive disease development.

1. A compound of formula I: 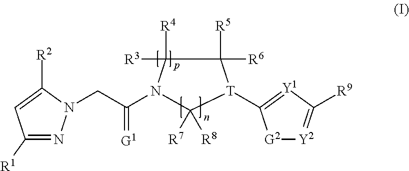
wherein,
G¹ and G² are independently O or S;
T is CR¹⁰ or N;
Y¹ and Y² are independently CR¹¹ or N;
n is 1 or 2;
p is 1 or 2, providing that when n is 2, p is 1;
R¹ and R² each independently are C₁-C₄alkyl, C₃-C₅cycloalkyl or C₁-C₄haloalkyl;
R³, R⁴, R⁵, R⁶, R⁷ and R⁸ each independently are hydrogen, C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl, providing that at least one of R³, R⁴, R⁵, R⁶, R⁷ or R⁸ is C₁-C₄alkylcarbonyl, C₃-C₅cycloalkylcarbonyl, C₁-C₄haloalkylcarbonyl, carboxyl, C₁-C₄alkoxycarbonyl, C₃-C₅cycloalkoxycarbonyl, C₁-C₄haloalkoxycarbonyl, aminocarbonyl, (C₁-C₄alkyl)aminocarbonyl, (C₃-C₅cycloalkyl)aminocarbonyl, (C₁-C₄haloalkyl)aminocarbonyl, (C₁-C₄alkyl)₂aminocarbonyl, (C₃-C₅cycloalkyl)₂aminocarbonyl or (C₁-C₄haloalkyl)₂aminocarbonyl;
R⁹ is C(═O)—OR¹², C(═O)—NR¹²R¹³ or a 4- to 15-membered mono-, bi- or tricyclic, saturated, partially unsaturated or aromatic heterocyclic ringsystem optionally substituted by one or more R¹⁴;
R¹⁰ is hydrogen, halogen or hydroxyl;
R¹¹ is hydrogen, halogen or cyano;
R¹² is arylalkyl, heteroarylalkyl or a 4- to 11-membered mono- or bicyclic, saturated, partially unsaturated or aromatic carbocyclic ringsystem, wherein the arylalkyl, heteroarylalkyl and 4- to 11-membered carbocyclic ringsystem are optionally substituted by one or more R¹⁴;
R¹³ is hydrogen, C₁-C₄alkyl, C₃-C₅cycloalkyl or C₁-C₄alkoxy;
each R¹⁴ independently is halogen, cyano, amino, nitro, hydroxyl, mercapto, C₁-C₈alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkoxy, C₃-C₈cycloalkyl-C₁-C₄alkylthio, C₁-C₈alkoxy, C₃-C₈cycloalkoxy, C₁-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkoxy, aryl-C₁-C₄alkylthio, heterocyclyl, heterocycyl-C₁-C₄alkyl, heterocycyl-C₁-C₄alkoxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R¹⁵; and
each R¹⁵ independently is halogen, cyano, C₁-C₄alkyl, C₁-C₄haloalkyl, C₁-C₄alkoxy or C₁-C₄haloalkoxy; or a salt or a N-oxide thereof.

2. A compound according to claim 1, wherein one of R³ and R⁵ is carboxyl, C₁-C₄alkoxycarbonyl, C₁-C₄haloalkoxycarbonyl, aminocarbonyl, (C₁-C₄alkyl)aminocarbonyl, (C₁-C₄haloalkyl)aminocarbonyl, (C₁-C₄alkyl)₂aminocarbonyl or (C₁-C₄haloalkyl)₂aminocarbonyl and R⁴, R⁶, R⁷ and R⁸ are hydrogen.

3. A compound according to claim 1, one of R³ and R⁵ is carboxyl or C₁-C₄alkoxycarbonyl and R⁴, R⁶, R⁷ and R⁸ are hydrogen.

4. A compound according to claim 1, wherein the compound of formula I is a compound of formula I-a 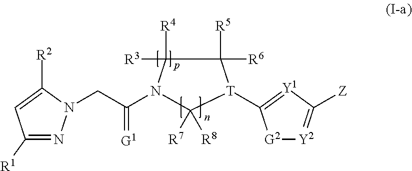
wherein Z is selected from Z-1 to Z-12 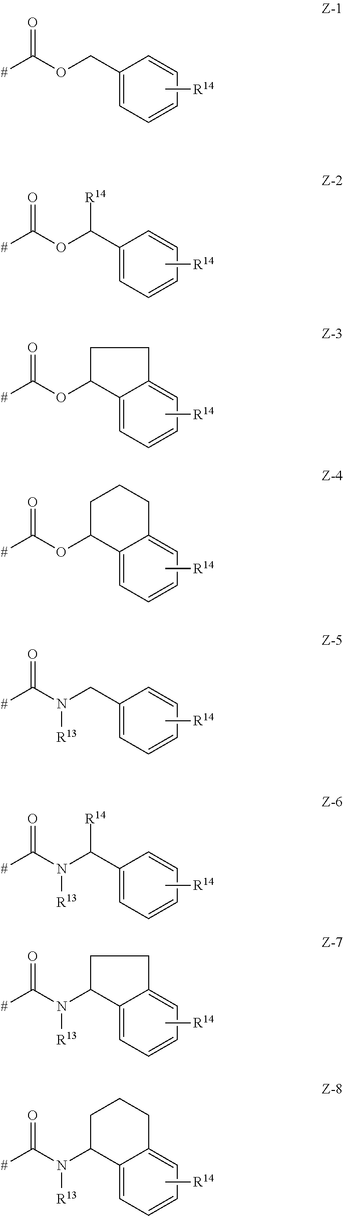 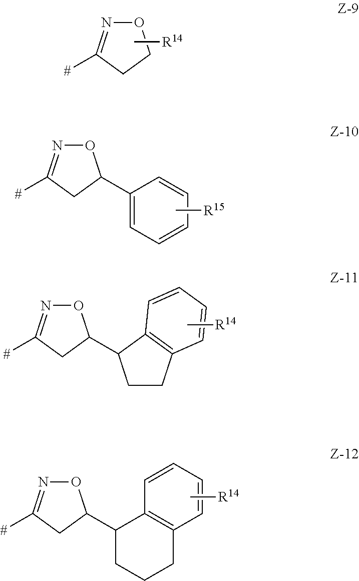
and G¹, G², T, Y¹, Y², n, p, R¹, R², R³, R⁴, R⁵, R⁶, R⁷, R⁸, R⁹, R¹⁰, R¹¹, R¹², R¹³, R¹⁴ and R¹⁵ are as defined for compound of formula I in claim 1.

5. A compound according to claim 4, wherein Z is selected from Z-1, Z-4, Z-8, Z-10 and Z-12.

6. A compound according to claim 4, wherein R¹⁴ and R¹⁵ are independently C₁-C₄alkyl, C₁-C₄haloalkyl or halogen.

7. A compound according to claim 1, wherein R¹ and R² are independently methyl or halomethyl.

8. A compound according to claim 1, wherein G¹ and G² are O.

9. A compound according to claim 1, wherein p is 1 and n is 2.

10. A compound of formula II 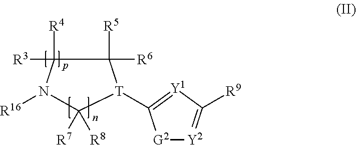
wherein R³, R⁴, R⁵, R⁶, R⁷, R⁸, R⁹, G², T, Y¹, Y², n and p are as defined for a compound of formula I in claim 1, and R¹⁶ is hydrogen or a protecting group, or a salt or N-oxide thereof, or
a compound of formula III 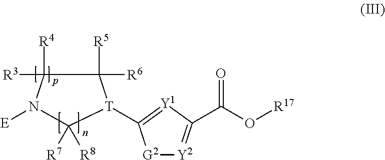
wherein E is hydrogen, a protecting group or group M 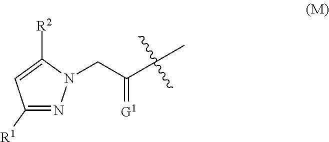
and R¹, R², R³, R⁴, R⁵, R⁶, R⁷, R⁸, G¹, G², T, Y¹, Y², n and p are as defined for a compound of formula I in claim 1, and R¹⁷ is C₁-C₆alkyl or a salt or N-oxide thereof, or
a compound of formula IV 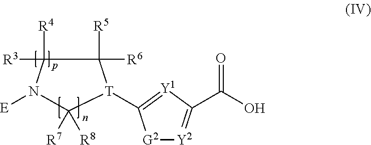
wherein E is hydrogen, a protecting group or group M 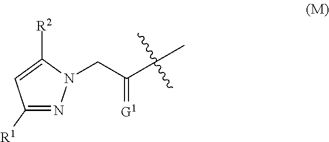
and R¹, R², R³, R⁴, R⁵, R⁶, R⁷, R⁸, G¹, G², T, Y¹, Y², n and p are as defined for a compound of formula I in claim 1, or a salt or N-oxide thereof, or
a compound of formula V 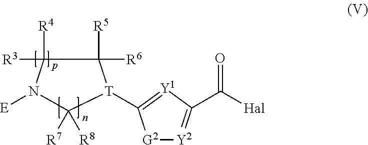
wherein E is hydrogen, a protecting group or group M, 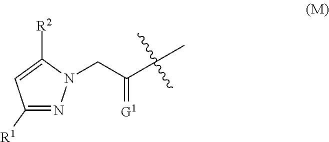
and R¹, R², R³, R⁴, R⁵, R⁶, R⁷, R⁸, G¹, G², T, Y¹, Y², n and p are as defined for a compound of formula I in claim 1, and Hal is halogen, preferably chloro or a salt or N-oxides thereof,
a compound of formula VI 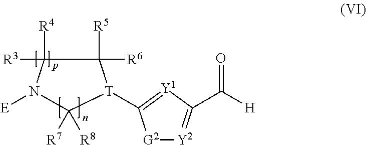
wherein E is hydrogen, a protecting group or group M 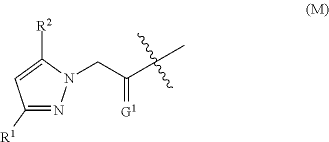
and R¹, R², R³, R⁴, R⁵, R⁶, R⁷, R⁸, G¹, G², T, Y¹, Y², n and p are as defined for a compound of formula I in claim 1, or a salt or N-oxides thereof,
a compound of formula VII 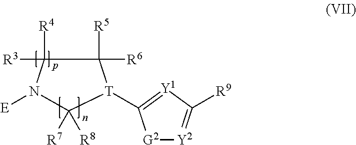
wherein E is hydrogen or a protecting group and R³, R⁴, R⁵, R⁷, R⁸, R⁹, G², Y¹, Y², n and p are as defined for a compound of formula I in claim 1, or a salt or N-oxides thereof.

11. A composition comprising at least one compound as defined in claim 1 and an agriculturally acceptable carrier, optionally comprising an adjuvant, and optionally comprising one or more additional pesticidally active compounds.

12. A method of controlling or preventing an infestation of plants, propagation material thereof, harvested crops or of non-living materials by phytopathogenic or spoilage microorganisms or organisms potentially harmful to man, which comprises the application of a compound as defined in claim 1, to the plant, to parts of the plants or to the locus thereof, to propagation material thereof or to any part of the non-living materials.
